(19) United States
(12) Patent Application Publication
(10) Pub. No.: US 2006/0019238 A1
(43) Pub. Date: Jan. 26, 2006

(54) Method for identifying protein-protein interactions

(75) Inventors: David M. Urech, Mannedorf (CH); Peter Lichtlen, Adliswil (CH); Alcide Barberis, Zurisch (CH)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ESBATech AG, Schlieren (CH)

(21) Appl. No.: 10/515,249

(22) PCT Filed: May 21, 2003
(86) PCT No.: PCT/EP03/05325

§ 371 Date: Jul. 8, 2005

Related U.S. Application Data
(60) Provisional application No. 60/382,774, filed on May 22, 2002.

(51) Int. Cl. C12Q 1/70; C12Q 1/68; G01N 33/53; C12P 21/06; C12N 9/12; G01N 33/567

(52) U.S. Cl. 435/5; 435/7.2; 435/6; 435/69.1; 435/194

(57) ABSTRACT

The properties of yeast help, a type I ER membrane protein which is involved in the unfolded protein response (UPR), have been exploited to develop â. system for the detection and study of interactions between extracellular and/or membrane proteins. In the system, proteins of interest are fused to the lumenal N-terminus of a truncated Ire1p. A specific interaction between two partners may be visualized through dimerization of the Ire1p moiety which, either, directly or indirectly, results in a detection means, for example, the expression of a selectable reporter gene. Depending on the type of reporter gene used, its expression can positively or negatively influence cell growth, thus allowing selection of both stimulation and inhibition of protein-protein interactions. The system presented here can also be used to study intracellular protein interactions.

### CROSS REFERENCE TO RELATED APPLICATIONS

This application claims priority from U.S. Provisional Application No. 60/382,774, filed May 22, 2002.

### BACKGROUND OF THE INVENTION

1. Field of the Invention

The present invention relates to a method for detecting the interaction of proteins using biological techniques.

2. Background of the Related Art

Methods for Identifying Protein-Protein Interactions

Protein-protein interactions provide the basis for critical and diverse biological functions. For example, transcription, DNA replication, enzyme regulation and assembly, antigen-antibody reactions and receptor-ligand systems all depend in some way on protein-protein interactions. It is also through protein-protein interactions that disease states and oncogenesis are perpetuated. It is, therefore, of interest to identify protein-protein interactions.

In addition to using well known biochemical techniques to study protein-protein interactions, a method for detecting protein-protein interactions using a genetic system has been described in U.S. Pat. No. 5,283,173 (hereby incorporated by reference). This two hybrid genetic system is capable of detecting proteins that interact with a known protein, determining which domains of the proteins interact, and providing the genes for newly identified interacting proteins. In that system, two hybrid proteins are constructed wherein one hybrid possesses a transcriptional activation domain linked to a first test protein and the other hybrid possesses a DNA binding domain linked to a second test protein. Therefore, in the two hybrid system of the '173 patent, interaction of the two test proteins results in formation of a viable transcription factor which can then activate a reporter gene. The protein-protein interaction and transcriptional activation both tale place in the nucleus of the yeast cell. Similar systems are described in U.S. Pat. No. 5,637,463 (hereby incorporated by reference). U.S. Pat. No. 5,503,977 (hereby incorporated by reference) describes an alternative system wherein an N-terminal subdomain and a C-terminal subdomain of ubiquitin are linked to a pair of proteins or peptides to be examined for their ability to interact and the subsequent cleavage at the quasi-native ubiquitin moiety within the linear protein fusion is the indication of interaction between the protein or peptide pair.

Many cell cycle regulatory proteins have been identified using yeast two-hybrid systems like the interaction trap (Gyuris et al., Cell 75:791, 1993; Harper et al., Cell 75:805, 1993; Serrano et al., Nature 366:704, 1993; Hannon et al., Genes & Dev. 7:2378, 1993). Typically, the interaction trap (Gyuris et al., supra) uses E. coli LexA repressor as the DNA-binding moiety and two different reporter genes, LEU2 and lacz, that each contain upstream LexA operators. Proteins that may interact with the bait, such as those encoded by members of cDNA libraries, are fused to an activation domain and expressed conditionally under the control of the yeast GAL1 promoter. To conduct an interactor hunt, cells that contain a bait are transformed with a library plasmid that expresses activation-tagged cDNA proteins, and transformants that contain proteins that associate with the bait are selected because they grow in the absence of leucine and form blue colonies on X-Gal medium. The most sensitive LEU2 reporter allows detection of interacting proteins with estimated K_{d}s less than 10⁻⁶ M (Gyuris et al., supra). Interacting proteins specific for the bait are identified as those that do not interact with unrelated baits.

These and other systems for identifying protein-protein interactions are useful in certain contexts, however, each has its own limitations. Therefore, new techniques which overcome any of these limitations represent important advances in the art.

The unfolded protein response

In eukaryotic cells, proteins that are destined for the cell surface or distal compartments are translocated and processed in the endoplasmic reticulum (ER), and then conducted through the secretory pathway to their final destination. The ER provides a unique oxidizing compartment in which a number of ER-resident chaperones facilitate the productive folding and the formation of disulfide bonds (for a review see [1]). Disulfide bonds between cystein residues strongly contribute to shape and stability of cell surface proteins [2]. In addition, (N)-linked glycosylation of proteins in the ER is a prerequisite for proper folding and can modulate the affinity of protein-protein interactions [3]. The environment present in the ER is, therefore, in marked contrast to the reducing environment of the cytosol which disfavors the formation of disulfide bonds. Another difference between the ER and the cytosol is that concentrations of Ca²⁺ are significantly higher in the ER than in the cytosol.

If proper protein maturation is impaired, unfolded or incorrectly folded proteins accumulate in the ER. Cells respond to this kind of stress by (a) stimulating transcription of genes encoding ER-resident chaperones and enzymes that assist protein folding and assembly in the ER lumen [3], and (b) increasing expression of members of the so-called ERAD ER-associated degradation) pathway [4]; [5], which leads to degradation of unfolded BR proteins. This so-called unfolded protein response (UPR) is common to all eukaryotes and presumes a communication between the BR lumen and the nucleus.

In Saccharomyces cerevisiae, the receptor that transmits the stress signal from the ER to the nucleus is the type 1 transmembrane protein Ire1p [6]. The N-terminal lumenal domain (NLD) of Ire1p is believed to control the dimerization function [7], whereas its C-terminal cytosolic part harbors a Ser/Thr protein kinase and an RNase domain. Dimerization of Ire1p brings its kinase domains in close proximity and leads to autophosphorylation in trans, which in turn activates its intrinsic endonuclease (Shamu et al. 1996, EMBO). It has been proposed that the ER-chaperone BiP binds the NIX of Ire1p, thus preventing dimerization and autophosphorylation in the absence of unfolded proteins: When unfolded proteins accumulate in the ER, BiP is titrated out by these proteins and dimerization of Ire1p can occur [7]. Dimerization of Ire1p is required for UPR signaling. In fact, substitution of the Ire1p NLD with a functional leucine zipper dimerization motif results in a constitutively active protein, thus indicating that dimerization or Ire1p may actually be the last check: point step in UPR signaling.

In an unconventional splicing reaction, sequential interaction of the activated endonuclease of the Ire1p dimer and the tRNA ligase remove a 252 nucleotide intron near the 3′ end of HAC1^{U} mRNA (“HAC1” for homology to ATF and CREB; “u” for UPR uninduced) to produce the HAC1ⁱ mRNA (“i” for UPR induced) [8], [9]. This splicing causes a change of the HAC1 open reading frame allowing synthesis of a functional protein, Hac1pⁱ. Hac1pⁱ is a DNA-binding protein with homology to the leucine zipper family of transcription factors. Upon activation of the UPR pathways; Hac1p binds to the unfolded protein response elements (UPRE) in the promoter region of ER-resident protein coding genes (such as KAR2) and thereby activates their expression ([10])(see FIG. 1). The UPRE is a single conserved 22-bp element (Mori et al, The Biology of Heat Shock Proteins and Molecular Chaperones, Cold Spring Harbor Press, pp. 417-55 (1992)). UPREs from different genes encoding ER resident proteins are characterized by short E box-like palindromic sequences separated by a single nucleotide (CANCNTG) (For Review, see Chapman et al, Annu. Rev. Cell Dev. Biol., 14:459-85 (1988) and references cited therein).

Two mutants of Ire1 have been described. [10] which can complement each other; Ire1K702R, which contains a point mutation in the kinase domain, and Ire1Δtail, a truncated form missing the last 133 amino acids of its C-terminus. While the Ire1K702R point mutation reduces the signaling potential of this protein to about 40%, Ire1Δtail shows no signalling activity.

Recently, homologs of the UPR have been identified in mammals and C. elegans (Yoshido, H. et al., Cell 107, 881-891 (2001) and Shen, X. et al., Cell 107, 893-903 (2001)). Mammalian cells have been found to express two Ire1p homologs designated as IRE1α and IRE1 β. Both are type 1 transmembrane proteins in the ER with their cytoplasmic regions comprising protein kinase and endoribonuclease domains. It has been shown that HAC1 precursor mRNA can be transfected into mammalian cells and is then correctly spliced in response to ER stress (Niwa et al., Cell 99, 691-702 (1999). Further, XBP1, a bZIP protein, has been shown to be processed by IRE1α in an ER-stressed cells in a manner highly analogous to the processing of Hac1 by Ire1. BiP has also been identified as part of the UPR in mammals. C. elegans has two homologs of mammalian BiP, HSP-3 and HSP-4, an Ire1 homolog (ire-1) and an XBP homolog (xbp-1). As can be appreciated, the UPR system is conserved in eukaryotes.

United States Patent Application U.S. 2002/0160408 A1 (“the '408 application”) discloses utilizing the IRE1 gene of yeast in a two-hybrid system. The application discloses in-reading frame fusions of ER proteins to the N-terminal “protein sensing domain” of IRE1p to detect their interaction using Ire1p dimerization and the unfolded protein response system as read out.

However, the prior art in general and the '408 application specifically fail to describe or suggest, for example, various advantageous read-out systems.

Accordingly, it is an object of the present invention to provide such methods or systems, the related components, and kits comprising them. Other deficiencies in the prior art will be evident in light of the disclosure below.

Single Chain Antibodies

Methods exist in the art for the identification of high-affinity binding single chain antibodies (i.e., scFV) using selection systems in an oxidizing environment such as phage display, mRNA display, ribosome display or immunization of mice (for example, Smith et al., Science 228: 1315-1317 (1985) and McCafferty et al., Nature 348: 552 (1990) describe phage display, Hanes et al., PNAS 94: 4937-4942 (1997) describes ribosome display; and Wilson et al., PNAS 98: 3750-3755 (2001) describe mRNA display). However these methods all have drawbacks, for example, by requiring purification of the antigen. This can be a laborious process. Therefore, a need exists in the art for a method of identifying high-affinity binding single chain antibodies which can be performed without the drawbacks of the prior art (i.e., the need for protein purification). Further, alternative methods would be useful simply as providing additional approaches for investigation of single chain antibodies.

Accordingly, it is an object of the present invention to apply the methods described herein in order to identify antigen-specific single-chain antibodies without the requirement of antigen purification and without the restriction to intracellular stability and solubility.

### BRIEF SUMMARY OF THE INVENTION

These and other objects are achieved by the present invention which provides a method and kit for detecting protein-protein interactions that occur either in the secretory pathway or in the extracellular or intracellular environment or, alternatively, detecting agents that inhibit protein-protein interactions in the secretory pathway or in the extracellular or intracellular environment.

It was discovered that a method could be designed to take advantage of the UPR cascade that is transmitted from the extracellular compartment to the nucleus. The method of the present invention takes advantage of one or more of the following: (a) the localization of Ire1p in the ER, (b) the dependence of Ire1 activity on dimerization and (c) the signaling pathway of Ire1 which results in the splicing dependent activation of Hac1p which then binds a defined sequence (UPRE) in the nucleus and activates transcription therefrom. Alternatively, a synthetic transcriptional activator may be used in place of Hac1p where the synthetic activator is also dependent on splicing for activation and the method is performed in a Hac1 minus background.

As will be described in more detail below, the method comprises substituting, for example, test proteins for the N-terminal lumenal domains of complementing Ire1 mutants. Interaction of the test proteins causes the dimerization of the complementing Ire1 mutants, the activation of the UPR cascade and, in turn, a signal to the user that the test proteins did, in fact, interact. This method allows the identification of extracellular or intracellular protein-protein interactions.

Alternatively, the test proteins may simultaneously interact with a ligand, where this binding causes the dimerization of the complementing Ire1 mutants, the activation of the UPR cascade and, in turn, a signal to the user that the test proteins did, in fact, interact with the ligand.

Advantages of the present invention include the ability to detect protein-protein interactions in the endoplasmic reticulum. This is an advantage because in cellular growth selection assays, all the cells in the neighborhood of a cell secreting a ligand which functionally interacts with a receptor would profit and thus grow, even if they express an unrelated ligand. Expression of the receptors and their soluble ligand in a closed compartment such as the ER, which provides the same properties as the extracellular space, should limit such background growth caused by the diffusion of the ligand.

Additionally, the use of the screening system described herein to find targets for extracellular protein-protein interactions, for example single chain antibodies, is a useful alternative to the phage display method, and provides the advantage of circumventing the need to purify target proteins. A single chain library fused to the C-terminus of Ire1p co-expressed with the fusion of a target protein to the C-terminus of Ire1p enables for the selection of proteins capable of binding the single chain antibody.

As will be appreciated by one of skill in the art, the conservation of the UPR in eukaryotes provides the opportunity to clone and express UPR components from one type of cell in another type of cell. For example, and as described above, the mammalian IRE1α may be used in a system which additionally comprises tide yeast mRNA Hac1.

### BRIEF DESCRIPTION OF THE DRAWINGS

FIG. 1:

The UPR signaling cascade in Saccharomyces cerevisiae: unfolded protein stress in the ER titrates out the chaperone BiP thus allowing dimerization of Ire1p. Dimerization-induced autophosphorylation of Ire1p activates its intrinsic endonuclease that cleaves the Hac1^{U}-mRNA. The resulting Hac1ⁱ-mRNA is translated into a functional Hac1p that translocates to the nucleus where, through its DNA binding domain (DBD), it binds UPRE's in the promoter regions of stress genes and, through its activation domain (AD), activates their expression.

FIG. 2:

One possible artificial UPR read out for use in the methods of the instant invention (also called “SCINEX-II” which stands for screening for intracellular and extracellular protein interactions). The LacZ reporter gene under the control of Hac1pⁱ allows quantification of the Ire1p activity. The HIS3 reporter gene enables growth selection of cells in which the UPR cascade has been activated. Other selectable genes can be used for a negative selection.

FIG. 3:

Map of constructs containing different moieties of Ire1p: “S” signal sequence, “NLD” N-terminal lumenal domain, “TM” transmembrane domain, “P” site of phosphorylation, “X” any protein moiety fused to the C-terminal of Ire1p, “M” myristoilation site (e.g. JunLZ, FosLZ, Ost¹⁻⁴⁴⁸, mEGFR-ECD, mFLT-1-ECD, mVEGF, mEGF). a) full length Ire1p. b) Ire1K702RΔNLD⁴⁹⁵, c) Ire1ΔtailΔNLDΔNLD⁴⁹⁵, d) Ire1K702RΔNLD⁵²⁶, e) Ire1ΔtailΔNLD⁵²⁶, f) Ire1ΔNLDΔTM, g) Mire1NLDΔTM.

FIG. 4:

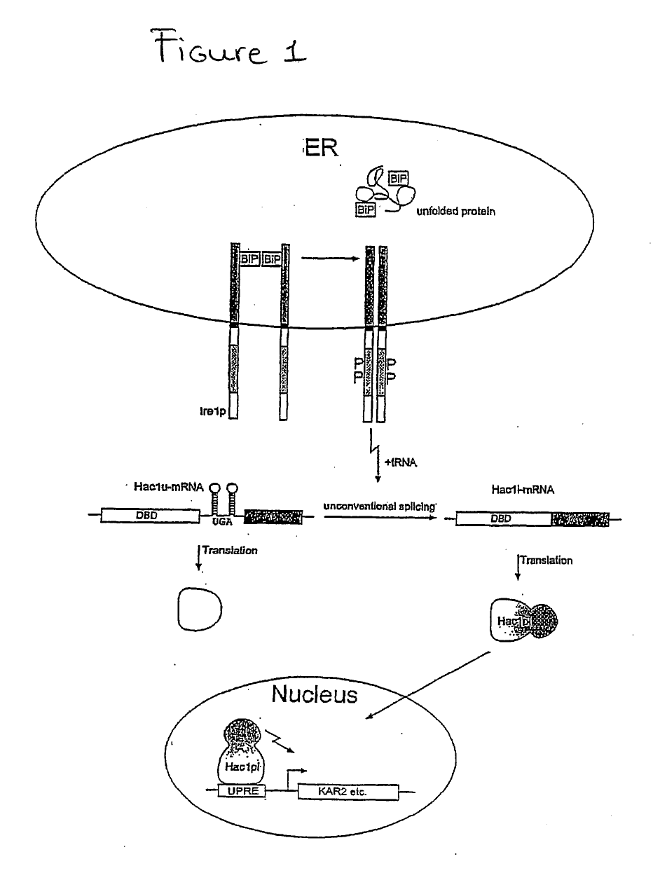

Quantification of UPR signaling by measuring the activity of the reporter gene product. β-Galactosidase. The constructs were expressed from ARS/CEN plasmids bearing either a TR1 or a LEU2 marker gene and grown on minimal medium lacking Trp and His. The highest value (line9) was set as 100%. White bars: cells which ex-press only one of the complementing Ire1p mutants; grey bars; cells expressing both complementing mutation of Ire1p but none or only one member of two interaction partners fused to the C-terminus of Ire1p; black bars: cells expressing both mutants fused to a pair of interaction partners.

FIG. 5:

Quantification of UPR signaling by measuring the activity of the reporter gene product β-Galactosidase. The constructs with either myristoilated JunLZ, not myristoilated JunLZ fused to Ire1ΔNLDΔTM or just the C-terminus of Ire1ΔNLDΔTM, were expressed from a ARS/CEN plasmid. Fusion proteins containing the JunLZ dimerization domain were active and further inducible with tunicamycine independently of the presence of the myristoilation domain. The Ire1p C-terminal fragment (which lacks the ability to dimerize) was instead inactive under both conditions.

FIG. 6:

Quantification of UPR signaling by measuring the activity of the reporter gene product β-Galactosidase. Constructs expressing a receptor fused to the Ire1K702RΔNLD⁵²⁶ were expressed from ARS/CEN plasmids with a LEU2 marker gene, those expressing a ligand fused to Ire1ΔtailΔNLD⁴⁹⁵ from ARS/CEN plasmids with a TRP1 marker gene. White bars: cells expressing only one of the dimeization partners; grey bars: cells expressing a ligand and an unrelated receptor; black bars: cells expressing a ligand and its fitting receptor.

FIG. 7:

Model of two possible applications of the SCINEX-II system for extracellular interactions: a) both interaction partners are fused to the Ire1p C-terminus. Dimerization and thus complementation leads to UPR signaling; b) soluble ligand is expressed in the secretory pathway where it binds its receptor and causes dimerization of the receptor chains. Localizing this action in the ER prevents that neighbouring cells profit from the diffusion of the ligand.

FIG. 8:

Assay for the interaction of three different single-chain antibodies directed against the leucine zipper of the yeast transcription factor GCN4 with antigen in the Ire1 system. Lane 1: Positive control: Jun-Jun-Dimers lead to activation of the Ire 1 system; Lane 2: Negative control: empty plasmids do not activate the system, Lane 3: The “Lambda graft” single chain fused to the point mutation of Ire 1, expressed in absence of the antigen does only mildly activate the system; Lane 4: The antigen “GCN4LZ” fused to the delta tail mutation of Ire 1, expressed in absence of any single chain antibody does not active the system; Lane 5: The antigen “GCN4LZ” fused to the delta tail mutation of Ire 1, co-expressed with the “Lambda graft” single chain, fused to the point mutation of Ire1 activates the system strongly and to a higher degree as when co-expressed, with the “kappa-graft” single chain (see lane 8), which has a lower affinity for the antigen according to in vitro measurement (see Wörn et al.); Lane 6: the GCN4 leucine zipper, when expressed as a fusion to both Ire1 mutants activates the system very strongly (as in nature the leucine zipper dimerizes with high affinity); Lane 7: The antigen “GCN4LZ” fused to the delta tail mutation of Ire 1, coexpressed with an unrelated protein (yeast Ost-1) fused to the point mutation of Ire 1 does only very mildly activate the system1; Lane 8: The antigen “GCN4LZ” fused to the delta tail mutation of Ire 1, co-expressed with the “Kappa graft” single chain, fused to the point mutation of Ire1 activates the system strong but to a lower degree as when co-expressed with the “lambda-graft” single chain (lane 5) or the “anti-GCN4”-single chain (lane 9), which have a higher affinity for the antigen according to in vitro measurement (see Wörn et al.); Lane 9: The antigen “GCN4LZ” fused to the delta tail mutation of Ire 1, co-expressed with the “anti-GCN4” single chain, fused to the point mutation of Ire1 activates the system strongly and to a higher degree as when co-expressed with the “kappa-graft” single chain (see lane 8), which has a lower affinity for the antigen according to in vitro measurement (see Wörn et al.). In addition, the “anti-GCN4” single chain is functional in this assay (which is not the case when it is expressed under the reducing intracellular conditions, see Wörnet al.).

FIG. 9

Epitope-scFv interaction-dependent UPRE reporter gene activation.

The Saccharomyces cerevisiae strain DIKU1-5 was transformed with Ars/Cen plasmids expressing the GCN4 leucine zipper epitope (GCN4LZ) and the different scfv's “λ-Graft”, “anti-GCN4”, “anti-GCN4(SS--)” and “AL-5”) fused to Ire1Δtail⁴⁹⁵⁻⁹⁸² and Ire1K702R⁴⁹⁵⁻¹¹¹⁵, respectively. The gene for the epitope-Ire1Δtail⁴⁹⁵⁻⁹⁸² fusion protein was expressed from a constitutive and strong actin promoter, while the genes encoding the scFv-Ire1K702R⁴⁹⁵⁻¹¹¹⁵ fusions were under the control of the weak IRE1 promoter. Binding of the various scFvs to the epitope was indirectly detected by measuring their ability to induce UPR signalling, and thus activate LacZ reporter gene transcription under the control of an UPRE (unfolded protein responsive element). LacZ reporter gene activity was quantified by measuring the enzymatic activity of β-Galactosidase. Transformants were incubated at 30° C. prior to assaying β-galactosidase activity. Expression of either the epitope or the scFvs alone did not result in a significant reporter gene induction. Co-expression of the epitope with the specific GCN4LZ binders “λ-Graft” or “anti-GCN4” strongly induced reporter gene activity. The non-specific “AL-5” and the mutated “anti-GCN4(SS--)” only slightly activated the system when co-expressed with GCN4LZ.

FIG. 10

Growth Selection of Epitope Binders.

Transformed saccharomyces cerevisiae cells were spotted in 1:5 dilution series with a starting concentration of 20000 cells/spot on synthetic complete agar plates lacking histidine, leucine, tryptophane with or without inositol and 0, 10 or 30 mM 3AT. These plates were incubated at 30° C. or 37° C. As an epitope, cells co-expressed the leucine zipper of GCN4 (GCN4LZ) fused to the Ire1 C-terminal moiety Ire1Δtail₄₉₅₋₉₈₂ and different single-chain Fvs (“λ-Graft”, “anti-GCN4”, “anti-GCN4(SS--) and “AL-5) fused to Ire1K702R⁴⁹⁵⁻¹¹¹⁵. A. IKU1-3 cells (ire1 Δ) expressing the GCN4LZ binding single-chain “λ-Graft” grew on selective conditions, while cells expressing the non-specific “AL-5” scFv were unable to grow on selective plates containing 30 mM 3AT. The most pronounced effect was observed when the epitope was expressed from the strong constitutive actin promoter and the scFvs from the very weal; Ire1 promoter. B. DIKU1-5 cells (ire1Δ; der1Δ) expressing one of the specific binders “λ-Graft” or “anti-GCN4” grew at every selective condition. In contrast, expression of the non-specific “AL-5” did not rescue growth at stringent conditions. While omitting inositol or incubation at 37° C. had a significant negative effect on growth only in the absence of any Ire1 derivative, the combination of incubating at 37° C. and the lack of inositol synergistically increased selectivity of the system. Addition of 30 mM 3AT further increased stringency. In contrast to the non-specific “AL-5”, which stopped growing on plates lacking inositol at 37° C., the mutated “anti-GCN4(SS--)” grew under these conditions. Since “anti-GCN4(SS--)” was selectable at 250 on plates lacking inositol and containing 30 mM 3AT, the most likely explanation for this apparent inconsistency is that this scFv, which is unable to form disulfide-bonds, probably tends to aggregate at elevated temperature and thus cause dimerization of Ire1K702R⁴⁹⁵⁻¹¹¹⁵, resultin in residual activity.

### DETAILED DESCRIPTION OF THE INVENTION

As will be appreciated by one of skill in the art, the conservation of the UPR system in eukaryotes provides the opportunity to utilize UPR components derived from many types of cells using techniques known to one of skill in the art. While the discussion herein may often refer to one particular system or set of proteins or mRNAs, such as those found in the yeast UPR system, it should be apparent that homologs from other eukaryotes may be used in similar ways and that such uses are contemplated in the instant invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

The term “chimeric” or “hybrid” protein is used to denote a protein or domain containing at least two component portions which are mutually heterologous in the sense that they do not occur together in the same arrangement in nature. More specifically, the component portions are not found in the same continuous polypeptide sequence or molecule in nature, at least not in the same order or orientation or with the same spacing present in the chimeric protein or composite domain.

The term test protein or fragment thereof refers to a protein or fragment that (i) does not occur in file Ire1 protein in nature; (ii) does not occur in the Ire1 protein in the same form in which it is present in the chimeric protein; or (iii) does not occur in nature with the same spacing that is present in the chimeric protein. In the most preferred embodiment, the test protein or fragment thereof is not related to the Ire1 protein.

The term “Ire1 derived polypeptide” or “Ire1 derived protein” as used herein refers to a polypeptide or protein which shares such homology or identity with Ire1 that it is capable of functioning as or substituting for native Ire1, with respect to the UPR pathway, as required by the methods of the instant invention. Specifically, the polypeptide would demonstrate that level of identity to Ire1 to be capable of functioning as required by the methods of the instant invention. In a preferred embodiment, this might mean that the polypeptide would exhibit 90%-100% identity with Ire1 when the portion of the Ire1 protein being used in the polypeptide is compared to the corresponding portion of the Ire1 protein. This could also mean that the polypeptide would exhibit 99% or greater identity. One of skill in the art will appreciate that a functional derivative, in light of the motivation provided herein and for purposes of the methods disclosed and described herein, may be devised using methods that are routine in the art and that such derivatives are contemplated in the instant invention.

The term “Ire1 homolog” as used herein refers to a protein that has the ability, when present as an activated dimer or heterodimer, to catalyze the splicing of a Hac1 homolog mRNA. For example, mammalian IRE1α or the C. Elegans ire-1 protein or yeast Ire1p are all Ire1 homologs.

The term “IRE1 like protein” as used herein refers to a protein that is either an Ire1 homolog or an Ire1 derived polypeptide. Such a protein would contribute to Ire1 like RNase activity when present as part of a complementing dimer.

The term “Hac1 mRNA homolog” as used herein refers to a mRNA that can be spliced by an activated dimer or heterodimer of an Ire1 homolog. For example, mammalian XBP-1 mRNA or the C. Elegans xbp-1 mRNA or the yeast Hac1p mRNA would be Hac1 mRNA homologs. Hac1 protein homolog could, accordingly, refer to the protein translated from a Hac1 mRNA homolog.

The term “Hac1 derived mRNA” as used herein refers to an mRNA that is a functional equivalent of Hac1 mRNA. A Hac1 derived mRNA could be either maintain the ability to be spliced or could also maintain the ability to be translated into a Hac1 derived polypeptide.

The term “Hac1 lice protein” or “Hac1 like mRNA” or “Hac1 like polypeptide” as used herein refers to a protein or mRNA or polypeptide, respectively, that is either a Hac1 homolog or Hac1 derived polypeptide or mRNA.

The term “introducing a DNA into the host cell” as used herein refers to the use of the methods described herein and those known to one of skill in the art for introducing DNA into appropriate host cells.

The term “subjecting the host cell to conditions” as used herein refers to maintaining or manipulating the appropriate conditions for the host cell for that given step, as would be known to one of skill in the art. In general, the term is used to describe those conditions that would be obvious to one of skill in the art and are also an element of routine experimentation.

The term “transcription factor Hac1” as used herein refers to the characterized transcription factor by that name or such variants that retain the function of Hac1 as required by the methods of the instant invention.

The term “yeast Hac1” may be used to refer to the transcription factor of that name and from that organism.

The term “identifying the chimeric genes” or “identifying the inhibiting agent” as used herein refers to, for example, any method for obtaining information regarding the amino acid sequence, DNA sequence, or chemical composition of the gene or agent. More specifically, the term “identifying the chimeric genes” refers to the process of; for example, isolating, sequencing or retrieving a chimeric gene from the host cell. Alternatively, the chimeric gene may be identified as a reagent used in a particular host cell and thus retrieved from storage etc. Regardless, the techniques involved in these processes are well known to one of skill in the art and represent routine experimentation.

The term “unfolded protein response element” or “UPRE” as used herein refers to a DNA sequence which can be specifically recognized by a HAC1 protein homolog. Consensus sequences for UPRE, methods of generating functional mutations of the UPRE, and methods of identifying additional sequences which are functionally equivalent to the UPRE are well known to one of skill in the art. UPREs would also include the endoplasmic reticulum response elements or ERSTs of mammalian cells. More specifically, yeast UPRE refers to, for example, a 22-bp element to which HAC1 protein is able to bind. As would be apparent to one of skill in the art, this binding sequence may be modified using known techniques to produce derivative sequences that would maintain binding ability. Such sequences wold also qualify as UPREs.

The term “yeast UPRE” as used herein refers to a DNA sequence which can be specifically recognized by the HAC1 protein. Consensus sequences for UPRE, methods of generating functional mutatations of the UPRE, and methods of identifying additional sequences which are functionally equivalent to the UPRE are well known to one of skill in the art. More specifically, UPRE refers to, for example, a specific 22-bp element from which HAC1 protein is able to activate expression. As would be apparent to one of skill in the art, this binding sequence may be modified using known techniques to produce derivative sequences that would maintain binding ability. Such sequences wold also qualify as UPREs.

The term “endoplasmic reticulum stress response element” or “ERSE” referes to a DNA sequence which can be specifically recognized by the XBP-1 protein. Consensus sequences for ERSE, methods of generating functional mutatations of the ERSE, and methods of identifying additional sequences which are functionally equivalent to the ERSE are well known to one of skill in the art. More specifically, mammalian ERSE refers to, for example, a specific cis-acting element from which XBP-1 protein is able to activate expression defined as CCAAT-N-9-CCACG. As would be apparent to one of skill in the art, this binding sequence may be modified using known techniques to produce derivative sequences that would maintain binding ability. Such sequences wold also qualify as ERSEs.

The term “signaling transcriptional activator” as used herein refers to an activator comprising the sequences necessary for splicing dependent translation by activated.

The term “synthetic transcriptional activator” as used herein refers to an activator comprising the sequences necessary for splicing dependent translation by activated Ire1 where that activator is not wild type Hac1.

The term “host cell” as used herein refers to any type of cell, including yeast, bacterial or mammalian cells. The preferred host cell is a yeast cell, preferably Saccharomyces cerivisiae.

The term “detectable gene” as used herein refers to any gene whose expression may be assayed. More than one detectable gene may be encoded by the host cell in the described embodiments. Examples of a detectable gene would be a gene which can be detected visually or through growth selection. Such genes are well known to one of skill in the art (i.e., HIS3, URA3, GFP etc.).

The term “signaling transcription factor” as used herein refers to a transcription factor capable of causing the expression of a detectable gene.

The term “signaling mechanism” as used herein refers to a mechanism capable of producing a visualizable or otherwise quantifiable result.

The term “Ire1 dimerization ability” refers to the ability of Ire1 to form dimers. This ability may be the result of a single domain or more than one domain may contribute to the dimerization ability.

According to one aspect of the present invention, there is provided a method for transferring a phosphate group to a first hybrid protein, the method comprising:
(a) providing a first chimeric gene that is capable of being expressed in the host cell; the first chimeric gene comprising a DNA sequence that encodes a first hybrid protein, the first hybrid protein comprising:
   (i) a first Ire1 like polypeptide with an inactive or absent native kinase domain; and
   (ii) a first test protein or fragment thereof that is to be tested for interaction with at least one second test protein or fragment thereof;
(b) providing a second chimeric gene that is capable of being expressed in the host cell, the second chimeric gene comprising a DNA sequence that encodes a second hybrid protein, the second hybrid protein comprising:
   (i) a second Ire1 like polypeptide which lacks the Ire1 dimerization ability but possesses a kinase domain; and
   (ii) a second test protein or fragment thereof that is to be tested for interaction with the first test protein or fragment thereof;
wherein interaction between the first test protein and the second test protein in the host cell results in the dimerization of the first hybrid protein and second hybrid protein, which results in transfer of a phosphate group to the first hybrid protein;
(c) introducing the first chimeric gene and the second chimeric gene into the host cell;
(d) subjecting the host cell to conditions under which the first hybrid protein and the second hybrid protein are expressed in sufficient quantity for the dimerization of the first hybrid protein and second hybrid protein; and
(e) subjecting the host cell to conditions under which the second hybrid protein catalyzes the transfer of a phosphate group to the first hybrid protein.

In a preferred embodiment, the host cell is a Hac⁻ cell that comprises a synthetic signaling transcription factor. In another preferred embodiment the host cell is both Ire1⁻ and ERAD⁻ and the cell is grown at elevated temperatures. In another preferred embodiment, the host cell is grown on media lacking inositol.

According to another aspect of the present invention, there is provided a method for transferring a phosphate group to a first hybrid protein, the method comprising:
(a) providing a first chimeric gene that is capable of being expressed in the host cell, the first chimeric gene comprising a DNA sequence that encodes a first hybrid protein, the first hybrid protein comprising:
   (i) an Ire1 like polypeptide with an inactive or absent native kinase domain; and
   (ii) a first test protein or fragment-thereof that is to be tested for interaction with at least one third test protein or fragment thereof;
(b) providing a second chimeric gene that is capable of being expressed in the host cell, the second chimeric gene comprising a DNA sequence that encodes a second hybrid protein, the second hybrid protein comprising:
   (i) an Ire1 like polypeptide which lacks the Ire1 dimerization ability but possesses a kinase domain; and
   (ii) a second test protein or fragment thereof that is to be tested for interaction with the third test protein or fragment thereof;
wherein a simultaneous interaction between the third test protein and both the first test protein and the second test protein in the host cell results in the dimerization of the first hybrid protein and second hybrid protein, which results in transfer of a phosphate group to the first hybrid protein;
(c) introducing the first chimeric gene and the second chimeric gene into the host cell;
(d) subjecting the host cell to conditions under which the first hybrid protein and the second hybrid protein and the third test protein are expressed in sufficient quantity for the dimerization of the first hybrid protein and second hybrid protein; and
(e) subjecting the host cell to conditions under which the second hybrid protein catalyzes the transfer of a phosphate group to the first hybrid protein.

According to another aspect of the present intention, there is provided a method for detecting an interaction between a first test protein and a second test protein, the method comprising:
(a) providing a host cell;
(b) providing a first chimeric gene that is capable of being expressed in the host cell, the first chimeric gene comprising a DNA sequence that encodes a first hybrid protein, the first hybrid protein comprising:
   (i) an Ire1 lice polypeptide with an inactive or absent native kinase domain; and
   (ii) a first test protein or fragment thereof that is to be tested for interaction with at least one second test protein or fragment thereof;
(c) providing a second chimeric gene that is capable of being expressed in the host cell, the second chimeric gene comprising a DNA sequence that encodes a second hybrid protein, the second hybrid protein comprising:
   (i) an Ire1 lice polypeptide which lacks the Ire1 dimerization ability but possesses a kinase domain; and
   (ii) a second test protein or fragment thereof that is to be tested for interaction with the first test protein or fragment thereof,
(d) introducing the first chimeric gene and the second chimeric gene into the host cell;
(e) subjecting the host cell to conditions under which the first hybrid protein and the second hybrid protein are expressed in sufficient quantity that the first hybrid protein and second hybrid protein dimerize and the second hybrid protein catalyzes the transfer of a phosphate group so the first hybrid protein wherein phosphorylation of the first hybrid protein results in a signal which can be detected.

In a preferred embodiment, the host cell is a Hac⁻ cell that comprises a synthetic signaling transcription factor. In another preferred embodiment the host cell is both Ire1⁻ and ERAD⁻ and the cell is grown at elevated temperatures. In another preferred embodiment, the host cell is grown on media lacking inositol.

According to another aspect of the present invention, there is provided a method for detecting an interaction between a first test protein and a second test protein, the method comprising:
(a) providing a host cell containing a detectable gene(s), wherein the detect-able gene(s) expresses a detectable protein(s) when the detectable gene(s) is activated by a signaling transcription factor, when the signaling transcription factor is in sufficient proximity to the detectable gene;
(b) providing a first chimeric gene that is capable of being expressed in the host cell, the first chimeric gene comprising a DNA sequence that encodes a first hybrid protein, the first hybrid protein comprising:
   (i) an Ire1 like polypeptide with an inactive or absent native kinase domain; and
   (ii) a first test protein or fragment thereof that is to be tested for interaction with at least one second test protein or fragment thereof;
(c) providing a second chimeric gene that is capable of being expressed in the host cell, the second chimeric gene comprising a DNA sequence that encodes a second hybrid protein, the second hybrid protein comprising:
   (i) an Ire1 like polypeptide which lacks the Ire1 dimerization ability but possesses a kinase domain; and
   (ii) a second test protein or fragment thereof that is to be tested for interaction with the first test protein or fragment thereof;
wherein interaction between the first test protein and the second test protein in the host cell results in the dimerization of the first hybrid protein and second hybrid protein which further results in the transfer of a phosphate group to the first hybrid protein catalyzed by the kinase domain of the second hybrid protein;
(d) introducing the first chimeric gene and the second chimeric gene into the host cell;
(e) subjecting the host cell to conditions under which the first hybrid protein and the second hybrid protein are expressed in sufficient quantity that the first hybrid protein and second hybrid protein dimerize; and
(f) subjecting the host cell to conditions under which the second hybrid protein catalyzes the transfer of a phosphate group to the first hybrid protein;
(g) subjecting the host cell to conditions under which phosphorylation of the first hybrid protein-results in activation of the signaling transcription factor;

(h) subjecting the host cell to conditions under which the activated signaling transcription factor is able to be in sufficient proximity to the detectable gene(s) to result in expression of the detectable protein(s);
(i) determining whether the detectable gene(s) has been expressed to a degree greater than expression in the absence of an interaction between the first test protein and the second test protein.

In a preferred embodiment, the host cell is a Hac⁻ cell that comprises a synthetic signaling transcription factor.

According to another aspect of the present invention, there is provided a method for identifying the DNA of interacting proteins, comprising performing steps (a)-(i) according to the above and further comprising:
(j) identifying the chimeric genes present in host cells which express the detectable gene to a degree greater than expression in the absence of an interaction between the first test protein and the second test protein.

According to a further embodiment of the invention, there is provided a method for identifying an inhibitor of an interaction between two proteins comprising:
(a) providing a host cell containing a detectable gene(s), wherein the detectable gene(s) expresses a detectable protein(s) when the detectable gene(s) is activated by a transcription factor Hac1, when the transcription factor is in sufficient proximity to the detectable gene;
(b) providing a first chimeric gene that is capable of being expressed in the host cell, the first chimeric gene comprising a DNA sequence that encodes a first hybrid protein, the first hybrid protein comprising:
   (i) an Ire1 derived polypeptide with an inactive or absent native kinase domain; and
   (ii) a first test protein or fragment thereof;
(c) providing a second chimeric gene that is capable of being expressed in the host cell, the second chimeric gene comprising a DNA sequence that encodes a second hybrid protein, the second hybrid protein comprising:
   (i) an Ire1 derived polypeptide which lacks the Ire1 dimerization ability but possesses a kinase domain; and
   (ii) a second test protein or fragment thereof wherein the first and second test proteins or fragments thereof interact;
wherein interaction between the first test protein and the second test protein in the host cell results in the dimerization of the first hybrid protein and second hybrid protein which further results in the transfer of a phosphate group to the first hybrid protein catalyzed by the kinase domain of the second hybrid protein;
(d) introducing the first chimeric gene and the second chimeric gene and an agent to be tested for possible inhibition into the host cell;
(e) subjecting the host cell to conditions under which the first hybrid protein and the second hybrid protein are expressed in sufficient quantity that the first hybrid protein and second hybrid protein could, in the absence of the inhibitor, dimerize; and
(f) subjecting the host cell to conditions under which the second hybrid protein could, in the absence of the inhibitor, catalyze the transfer of a phosphate group to the first hybrid protein;
(g) subjecting the host cell to conditions under which any phosphorylation of the first hybrid protein would result in activation of the transcription factor Hac1p;
(h) subjecting the host cell to conditions under which the activated transcription factor Hac1p is able to be in sufficient proximity to the detectable gene(s) to result in expression of the detectable protein(s);
(i) determining whether the detectable gene(s) has been expressed to a degree less than expression in the absence of the agent;
(j) identifying the agent used as the inhibitor when the detectable gene has been expressed to a degree less than expression in the absence of the agent.

As is apparent from the above, in one embodiment the test proteins may simultaneously interact with at least a third protein or ligand, where this binding causes the dimerization of the complementing Ire1 mutants, the activation of the UPR cascade and, in turn, a signal to the user that the test proteins did, in fact, interact with the ligand Such a method may be used to screen for single chain antibodies which bind antigen with high affinity under physiological oxidizing conditions in vivo, for example by screening a CDR-randomized single-chain antibody library. Such an approach may be an attractive alternative to conventional phage display.

In that respect, in a preferred embodiment, the third test protein is a single chain antibody. For example, two Ire1 complementing mutants can be fused to protein A and protein B, respectively where protein A and protein B do not directly interact. A single chain antibody capable of binding protein A and protein B simultaneously will result in the dimerization of the complementing Ire1 mutants. Alternatively, a single chain antibody may be screened based on its ability to disrupt interaction between two proteins. For example, interacting proteins C and D are each fused to complementing Ire1 mutants. A scFV which interacts with protein C and disrupts the interaction between C and D can be identified based on loss of signal.

In another embodiment, a soluble ligand may be used as a third protein and the Ire1 complementing mutants may be fused to the receptor.

As would be known by one of skill in the art, any of the methods described for transferring a phosphate group to a first hybrid protein may be used in the methods for detecting the protein-protein interactions. In that respect, method steps may be clearly interchangeable and such methods are contemplated herein.

In addition to these methods, embodiments of the invention include the chimeric genes, chimeric proteins, vectors, and host cells utilized in the methods and kits comprising any or all of the components used in the methods.

In a preferred embodiment of the invention, the host cell is selected from the group consisting of Saccharomyces cerevisiae, mammalian cells, eukaryotic cells; and prokaryotic cells.

In a preferred embodiment of the invention, the first hybrid protein or the second hybrid protein is encoded on a library of plasmids containing DNA inserts, derived from the group consisting of genomic DNA, cDNA and synthetically generated DNA.

In a preferred embodiment of the invention, the first test protein or second test protein or both the first and second test proteins are derived from the group consisting of bacterial proteins, viral proteins; oncogene-encoded proteins, eukaryotic proteinsplant proteins, yeast proteins, orphan receptors, antibodies, antigens, ligands, any transmembrane protein, any cell surface protein, any extracellular protein, any protein expressed in the secretory pathway, and any intracellular protein.

In a preferred embodiment of the invention, the chimeric genes are introduced into the host cell in the form of plasmids.

In a preferred embodiment of the invention, the first chimeric gene is integrated into the chromosomes of the host cell.

In a preferred embodiment of the invention, the first chimeric gene is integrated into the chromosomes of the host cell and the second chimeric gene is introduced into the host cell as part of a plasmid.

In a preferred embodiment of the invention, the Ire1 like polypeptide is selected from the group consisting of Ire1 homologs, Ire1 derived polypeptides and Ire1 polypeptides.

In a preferred embodiment of the invention, the Ire1 like polypeptide with the inactive or absent native kinase domain is any complementable kinase mutant of Ire1.

In a preferred embodiment of the invention, the Ire1 derived polypeptide with the inactive or absent native kinase domain is selected from the group consisting of Ire1K702R, Ire1 K702RΔNLD⁴⁹⁵, Ire1 K702RΔNLD⁵²⁶, Ire1K702RΔNLDΔTM, Myristoylated Ire1 K702RΔNLDΔTM and any fragment or derivative of these capable of complementing an Ire1 mutant which lacks dimerization ability.

In a preferred embodiment of the invention, the Ire1 derived polypeptide which lacks the Ire1 dimerization ability but possesses a kinase domain is any complementable dimerization mutant of Ire1.

In a preferred embodiment of the invention, the Ire1 derived polypeptide which lacks the Ire1 dimerization ability but possesses a kinase domain is selected from the group consisting of Ire1Δtail, Ire1ΔtailΔNLD⁴⁹⁵, Ire1ΔtailΔNLD⁵²⁶, Ire1 tailΔTM, myristoylated Ire1tailΔTM and any fragment or derivative of these capable of complementing an Ire1 mutant which lacks the dimerization ability.

In a preferred embodiment of the invention, the interaction between the first test protein and second test protein occurs in the cytoplasm, on the cell surface or anywhere in the secretory pathway.

In a preferred embodiment of the invention, either the first test protein or the second test protein or both the first test protein and the second test protein are expressed such that they remain in the endoplasmic reticulum.

In a preferred embodiment of the invention, either the first test protein or the second test protein or both the first test protein and the second test protein are full length proteins.

In a preferred embodiment of the invention, either the first test protein or the second test protein or both the first test protein and the second test protein possess transmembrane domains.

In a preferred embodiment of the invention, either the first test protein or the second test protein is a single chain antibody.

In a preferred embodiment of the invention, the detectable gene is the LacZ gene.

In a preferred embodiment of the invention, the detectable gene is the HIS3 gene.

In a preferred embodiment of the invention, the detectable genes are the LacZ gene and the HIS3 gene.

In a preferred embodiment of the invention, the detectable gene is selected from the group consisting of CAT (chloramphenicol acetyltransferase), GAL (β-galactosidase), GUS (β-glucuronidase), LUC (luciferase), and GFP (green fluorescent protein). Additional reporter genes are comprised in the skill of the art and are contemplated in this invention.

In a preferred embodiment of the invention, the detectable gene is in proximity to an Unfolded Protein Response Element (UPRE).

In a preferred embodiment of the invention, the UPRE is the yeast UPRE.

In a preferred embodiment of the invention, the UPRE is an ERST.

According to another aspect of the present invention, there is provided a chimeric gene comprising a DNA sequence that encodes a hybrid protein, the hybrid protein comprising: an Ire1 like polypeptide with an inactive or absent native kinase domain and a test protein, or fragment thereof.

According to another aspect of the present invention, there is provided a chimeric gene comprising a. DNA sequence that encodes a hybrid protein, the hybrid protein comprising an Ire1 like polypeptide which lacks the Ire1 dimerization ability but possesses a kinase domain and a test protein or fragment thereof.

In a preferred embodiment of the invention, the Ire1 lice polypeptide is selected from the group consisting of Ire1 homolog polypeptides, Ire1 derived polypeptides, and Ire1 polypeptides.

In a preferred embodiment of the invention, the Ire1 like polypeptide is any complementable kinase mutant of Ire1.

In a preferred embodiment of the invention, the Ire1 like polypeptide is selected from the group consisting of Ire1K702R, Ire1 K702RΔNLD⁴⁹⁵, Ire1 K702RΔNLD⁵²⁶, Ire1 K702RΔNLDΔTM, Myristoylated Ire1 K702RΔNLDΔTM and any fragment or derivative of these capable of complementing an Ire1 mutant which lacks the dimerization ability.

In a preferred embodiment of the invention, the Ire1 lice polypeptide is any complementable dimerization mutant of Ire1.

In a preferred embodiment of the invention, the Ire1 like polypeptide is selected from the group consisting of Ire1Δtail, Ire1ΔtailΔNLD⁴⁹⁵, Ire1ΔtailΔNLD⁵²⁶, Ire1ΔtailΔTM, myristoylated Ire1tailΔTM, any fragment or derivative of these capable of complementing an Ire1 mutant which lacks dimerization ability.

According to another aspect of the present invention, there is provided a protein encoded by a chimeric gene of the instant invention.

According to another aspect of the present invention, there is provided a vector comprising a chimeric gene of the instant invention.

According to another aspect of the present invention, there is provided a vector comprising a DNA sequence capable of encoding an Ire1 like polypeptide wherein the native kinase domain of the polypeptide is inactive or absent and further comprising a cloning site which allows for the construction of the chimeric gene.

According to another aspect of the present invention, there is provided a vector comprising a DNA sequence capable of encoding an Ire1 like polypeptide wherein the polypeptide lacks the Ire1 dimerization ability but possesses a kinase domain and further comprising a cloning site which allows for the construction of a chimeric gene.

According to another aspect of the present intention, there is provided a host cell comprising any of the chimeric genes of the instant invention.

According to another aspect of the present invention, there is provided a kit comprising any one or more of a chimeric gene, a vector and a host cell.

According to another aspect of the present invention, there is provided a method for identifying an inhibitor of an interaction between two proteins comprising:
(a) providing a host cell;
(b) providing a first chimeric gene that is capable of being expressed in the host cell, the first chimeric gene comprising a DNA sequence that encodes a first hybrid protein, the first hybrid protein comprising:
   (i) an Ire1 like polypeptide with an inactive or absent native kinase domain; and
   (ii) a first test protein or fragment thereof that is to be tested for interaction with at least one second test protein or fragment thereof;
(c) providing a second chimeric gene that is capable of being expressed in the host cell, the second chimeric gene comprising a DNA sequence that encodes a second hybrid protein, the second hybrid protein comprising:
   (i) an Ire1 like polypeptide which lacks the Ire1 dimerization ability but possesses a kinase domain; and
   (ii) a second test protein or fragment thereof that is to be tested for interaction with the first test protein or fragment thereof;
(d) introducing the first chimeric gene and the second chimeric gene and an inhibitor candidate into the host cell;
(e) subjecting the host cell to conditions under which the first hybrid protein and the second hybrid protein are expressed in sufficient quantity that the first hybrid protein and second hybrid protein could, in the absence of an inhibitor, dimerize wherein dimerization would cause the second hybrid protein to catalyze the transfer of a phosphate-group to the first hybrid protein wherein phosphorylation of the first hybrid protein results in a sigal which can be detected;
(i) determining whether the signal is stronger or weaker than the signal in the absence of the agent; and
(j) identifying the agent used as the inhibitor when the detectable gene has been expressed to a degree less than expression in the absence of the agent.

As would be apparent to one of skill in the art, many of the methods disclosed herein may be manipulated for use in screening assays designed to identify inhibitors. Such assays are contemplated by this invention.

In a preferred embodiment of the invention, the agent is selected from the group consisting of proteins, small molecules, chemical compounds, peptides and natural molecules.

In a preferred embodiment of the invention, the signal comprises a signaling transcription factor interacting with a detectable gene.

In a preferred embodiment of the invention, the signaling trancription factor is a Hac1 like polypeptide.

In a preferred embodiment of the invention, the transcription factor is a synthetic transcriptional activator.

In a preferred embodiment of the invention, the Ire1 like polypeptides are selected from the group consisting of Ire1 homolog polypeptides, Ire1 derived polypeptides and Ire1 polypeptides.

In a preferred embodiment of the invention, the synthetic transcriptional activator is translated from RNA that is spliced by Ire1 like RNase activity.

In a preferred embodiment of the invention, the host cell does not express endogenous Hac1 like polypeptides.

In a preferred embodiment of the invention, the host cell does not produce endogenous Ire1 like polypeptides.

In a preferred embodiment of the invention, the host cell does not produce endogenous Ire1, like polypeptides.

In a preferred embodiment of the invention, the host cell used in the method is Saccharomyces cerivisiae.

In a preferred embodiment of the invention, the hybrid proteins are encoded on a library of plasmids containing DNA inserts.

In a preferred embodiment of the invention, the test protein is a receptor, ligand, or antibody.

In a preferred embodiment of the invention, the chimeric genes are introduced into the host cell in the form of plasmids.

In a preferred embodiment of the invention, the chimeric gene or genes are integrated into the host chromosome.

In a preferred embodiment of the invention, the Ire1 derived polypeptide with the inactive or absent native kinase domain is Ire1K720R

In a preferred embodiment of the invention, the Ire1 derived polypeptide which lacks the Ire1 dimerization ability but possesses a kinase domain is Ire1 Δtail.

In a preferred embodiment of the invention, the interaction between the first test protein and second test protein occurs in the endoplasmic reticulum or the cytoplasm.

In an alternate embodiment of the invention, the first or second or both the first and second test proteins are attached to endoplasmic reticulum retention signals or transmembrane domains.

In a preferred embodiment, the first or second test protein is a single chain antibody.

In a preferred embodiment of the inventions the detectable gene is LacZ or HIS3 or both.

In one embodiment, the signaling transcription factor is a synthetic transcription factor. In another embodiment, the signaling transcription factor is Hac1.

In a preferred embodiment, the promoter for the detectable gene is an unfolded protein response element (UPRE).

A further preferred embodiment is directed towards the chimeric gene, wherein the chimeric gene is a gene capable of encoding any of the hybrid proteins of the described embodiments.

A further preferred embodiment is directed towards the protein encoded by the protein encoded by a chimeric gene, wherein the chimeric gene of the invention.

A further preferred embodiment is a vector comprising the chimeric gene of the invention.

A further embodiment is a host cell comprising any one or more of the chimeric genes of the invention.

A further embodiment of the invention is a kit comprising any of the components described herein.

A further embodiment of the invention is the use of the methods and systems described herein for the identification of agents capable of inhibiting the interaction of proteins.

In a preferred embodiment, the inhibitory agent is a small molecule or chemical compound or peptide or antibody or protein.

In one embodiment, determining whether the detectable gene has been expressed to a degree lesser than or greater than the expression in a control cell may be done, for example, by monitoring growth of the cell on a nutritionally deficient growth medium wherein the interacting proteins cause transcription of a biosynthetic gene or pathway. Examples of useful detectable means include amino acid, metabolic, catabolic and nucleic acid biosynthetic genes, such as yeast HIS3, UBA3, and LYS3, GAL1, E. coli galK and CAT, GUS, antibiotic resistance, and any gene encoding a cell surface antigen for which antibodies are available. The cell may be allowed to grow for any period of time determined by one of skill in the art to be appropriate, for example, from 3-10 days.

In another embodiment, the signal may simply be the accumulation of processed or spliced mRNA or any other type of signal which results from the dimerization of the Ire1 like polypeptides and may be quantified.

Recently, it was found that the UPR regulates not only the expression of chaperones and enzymes that assist folding, but also members of the ERAD, which are involved in degrading unfolded ER proteins (Travers K. J. et al., 2000). Double knock-out cells for both Ire1p and the ERAD genes DER1, HRD1 or BRD3 are temperature sensitive (Travers K. J. et al., 2000). Therefore, in one embodiment, such double knock-out cells provide an alternative or more stringent read-out system. Double knock-out cells expressing C-terminal fragments of the Ire1 complementing mutants fused to proteins that interact with each other, thus mimicking endogenous Ire1p activity, should grow at elevated temperatures. Cells expressing proteins that do not interact should not grow at the non-permissive temperature. In a preferred embodiment, such a system could additionally be used in combination with a transcriptional read-out system, as described herein, to create a very stringent selection system.

In another embodiment, a read out system is devised utilizing the mRNA of a synthetic transcription activator containing the Hac1 intron and other sequences necessary for the splicing reaction performed by Ire1p and tRNase. In addition, by selecting a suitable reporter gene, growth selection of either agonists or antagonists can be performed. Such techniques would be well known to one of skill in the art.

In another embodiment, the read out system is devised based on the knowledge that cells lacking Ire1p or Hac1p require inositol for growth (Cox, J. et al., Cell 73, 1197 (1993); Mori, K. et al., Cell 74, 743-756 (1993); Cox, J. S. et al., Cell 87, 391-404 (1996); Sidrauski, C. et al., Cell 87, 405413 (1996)). In that respect, growth selection on inositol lacking media could be used and growth would be the signal which can be detected.

In addition, Ire1 phenotypes which are not dependent on the activation of a transcription factor through splicing may be envisioned For example, the ire1der1 double knockout is temperature sensitive. In this strain, a reconstitution of Ire1 by dimerization of the complementing mutants would rescue cell growth at elevated temperatures, thus providing the required detecting means for design of the method.

This invention further provides, in one embodiment, kits useful for the foregoing applications. One such kit contains a first and second DNA sequence encoding a chimeric protein of this invention and a third DNA sequence containing a target gene linked to a DNA sequence capable of being bound by a downstream transcription-factor activated as part of a cascade response to dimerization of polypeptides encoded by the first and second DNA sequences. Alternatively, the third DNA sequence may contain a cloning site for insertion of a desired target gene by the practitioner. In general, such kits may comprise any one or more of the individual components of the methods described herein by themselves or in combination, for example, with other useful reagents for conducting any step or steps of the methods described herein, apparatus useful for conducting any step or steps herein, or in combination with instructions or other packaging.

Those skilled in the art will recognize that the detectable gene or reporter gene may be derived from any appropriate eukaryotic or prokaryotic cell gnomes or cDNAs as well as artificial sequences. Moreover, although yeast represents a preferred host, other hosts such as mammalian cells may be used.

Using DNA sequences encoding the chimeric proteins of this invention, and vectors capable of directing their expression in eukaryotic cells, one may genetically engineer cells for a number of important uses. To do so, one first provides an expression vector or construct for directing the expression in a eukaryotic cell of the desired chimeric protein and then introduces the vector DNA into the cells in a manner permitting expression of the introduced DNA in at least a portion of the cells. One may use any of the various methods and materials for introducing DNA into cells for heterologous gene expression, many of which are well known. A variety of such materials are commercially available.

DNA sequences encoding individual domain(s) or sub-domain(s) and linkers, if any, are joined such that they constitute a single open reading frame encoding a chimeric protein containing, for example, the Ire1 derived region and capable of being translated in cells or cell lysates into a single polypeptide harboring all component domains. This protein-encoding DNA sequence is then placed into a conventional plasmid vector that directs the expression of the protein in the appropriate cell type. For testing of proteins and determination of protein-protein interactions, it may be desirable to construct plasmids that direct the expression of the protein in bacteria or in reticulocyte-lysate systems. For use in the production of proteins in mammalian cells, the protein-encoding sequence is introduced into an expression vector that directs expression in these cells. Expression vectors suitable for such uses are well known in the art. Various sorts of such vectors are commercially available.

This invention further encompasses, in one embodiment, genetically engineered cells containing and/or expressing any of the constructs described herein, particularly a construct encoding a chimeric protein of the instant invention, including prokaryotic and eucaryotic cells and in particular, yeast, worm, insect, mouse or other rodent, and other mammalian cells, including any human cells, of various types and lineages, whether frozen or in active growth, whether in culture or in a whole organism containing them. Several examples of such engineered cells are provided in the Examples which follow. Those cells may further contain a DNA sequence to which the encoded chimeric protein is capable signaling either directly or as part of a cascade. Likewise, this invention encompasses any non-human organism containing such genetically engineered cells.

In a transient transfection assay, the above-mentioned plasmids are introduced together into tissue culture cells by any conventional transfection procedure, including for example calcium phosphate coprecipitation, electroporation, and lipofection. After an appropriate time period, usually 24-48 hr, the cells are harvested and assayed for production of the reporter or detectable protein. In an appropriately designed system, the reporter gene should exhibit little activity above background in the absence of any Ire1 kinase activity. In contrast, reporter gene expression should be elevated in a dose-dependent fashion by the inclusion of plasmids encoding the chimeric proteins which result in Ire1 kinase activity. This result indicates that there are few natural transcription factors in the recipient cell with the potential to recognize the tested binding site and activate transcription and that the transcription factor activated by Ire1 kinase activity is capable of binding to this site inside living cells. In the preferred embodiment, the transcription factor activated by Ire1 kinase activity is Hac1.

Plasmid constructs, transformation, transfection, cell culture and detection of transcription may be performed by any method known in the art, for example, U.S. Pat. No. 5,283,173 and WO 94/10300 and U.S. Pat. No. 6,332,897. Any means for introducing genes into host cells may be used, for example, electroporation, transfection, and transformation.

Constructs encoding the chimeras of the instant invention and constructs directing the expression of target genes, all as described herein, can be introduced into cells as one or more DNA molecules or constructs, in many cases in association with one or more markers to allow for selection of host cells which contain the construct(s). The constructs can be prepared in conventional ways, where the coding sequences and regulatory regions may be isolated, as appropriate, ligated, cloned in an appropriate cloning host, analyzed by restriction or sequencing, or other convenient means. Particularly, using PAR, individual fragments including all or portions of a functional unit may be isolated, where one or more mutations may be introduced using “primer repair”, ligation, in vitro mutagenesis, etc. as appropriate. The construct(s) once completed and demonstrated to have the appropriate-sequences may then be introduced into a host cell by any convenient means. The constructs may be integrated and packaged into non-replicating, defective viral genomes like Adenovirus Adeno-associated virus (AAV), or Herpes simplex virus (HSV) or others, including retroviral vectors, for infection or transduction into cells. The constructs may include viral sequences for transfection, if desired. Alternatively, the constuct may be introduced by fusion, electroporation, biolistics, transfection, lipofection, or the like. The host cells will in some cases be grown and expanded in culture before introduction of the construct(s), followed by the appropriate treatment for introduction of the construct(s) and integration of the construct(s). The cells will then be expanded and screened, for example, by virtue of a marker present in the construct. Various markers which may be used successfully include hprt, neomycin resistance, thymidine kinase, hygromycin resistance, etc.

In some instances, one may have a target site for homologous recombination, where it is desired that a construct be integrated at a particular locus. For example, one can delete and/or replace an endogenous gene (at the same locus or elsewhere) with a recombinant target construct of this invention. For homologous recombination, one may generally use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding all of the genes, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in procaryotes or eucaryotes, etc. which may be used to prepare stocks of construct DNAs and for carrying out transfections are well known in the art, and many are commercially available.

Cells which have been modified ex vivo with the DNA constructs may be grown in culture under selective conditions and cells which are selected as having the desired construct(s) may then be expanded and further analyzed, using, for example, the polymerase chain reaction for determining the presence of the construct in the host cells. Once modified host cells have been identified, they may then be used as planned, e.g. grown in culture or introduced into a host organism.

The invention may be illustrated by the following examples which are not intended to limit the scope of the invention in any way.

### EXAMPLE 1

The NLD of the Ire1 complementing mutants was substituted with known interacting partners in order to make the induction of the UPR pathway and consequent reporter gene expression dependent on a specific interaction happening either in the ER-lumen or in the cytoplasm.

Depending on the original location of the studied proteins, the respective Ire1p fusions were expressed either in the ER or in the cytoplasm.

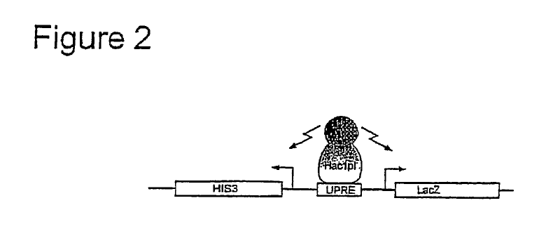

The activated Hac1pⁱ induces expression of two selectable reporter genes, HIS3 and LacZ that bear a UPRE sequence upstream of their divergent promoters (FIG. 2).

Cloning of the Ire1 Fusions

IRE1 DNA sequences were amplified from yeast genomic DNA by PCR with proof-start polymerase (QIAGEN) using primers that contained restriction sites at their 5′ end. To generate the Ire1K702R point mutation, two additional primers harbouring a base-pair change were used to amplify a 5′ fragment and a 3′ fragment of the Ire1 C-terminus, each harbouring the respective base-pair change. The two fragments were ligated by assembled PCR resulting in the complete Ire1 C-terminus containing the K702R point mutation. Different regions of IRE1 were amplified to generate the following Ire1p fragments: Ire1ΔNLD⁴⁹⁵ wild type Ire1 C-terminus extending from amino acid 495 to 1115; Ire1K702RΔNLD⁴⁹⁵: the same part of Ire1 as in Ire1ΔNLD⁴⁹⁵, but harbouring a point mutation in the kinase domain; Ire1ΔtailΔNLD⁴⁹⁵ Ire1 C-terminus extending from amino acid 495 to 982, lacking its very C-terminal tail. Truncated versions were amplified using the fragments mentioned above as templates. Namely Ire1 C-termini lacking their complete NLD referred to as Ire1ΔNLD⁵²⁶ Ire1K702RΔNLD⁵²⁶, Ire1ΔtailΔNLD⁵²⁶ and Ire1 C-termini lacking their NLD and their transmembrane domain termed as Ire1ΔNLDΔTM. All the primers binding to the 5′ part of the Ire1 C-terminus contained a NotI site.

The DNA sequence encoding the mouse EGF receptor extracellular domain (ECD) was amplified from a mouse liver cDNA library by nested PCR. The mouse DNA sequence encoding the VEGF receptor mFLT-1 ECD was amplified by RT-PCR from mouse embryonic RNA. The mouse VEGF gene was amplified from a mouse embryonic cDNA library. In a second round of PCR using the first products as template, the signal sequences of the amplified coding sequences were substituted with the signal sequence of the Suc2 gene of Saccharomyces cerevisiae by using primers containing a Suc2 signal sequence at their 5′ end. The sequence expressing the lumenal part of Ost1¹⁻¹⁴⁸ was amplified from yeast genomic DNA. To obtain the mouse EGF, we performed genesynthesis: four overlapping oligos where ligated by assembled PCR. All the primers assembling at the 3′ end of the coding sequence of the genes fused to Ire1 contained a NotI restriction site. This allowed in-frame fusion to the Ire1 C-termini leading to the following junction: ECD-ggc ggc cgc-Ire1 (NotI site bold). The fusion proteins were expressed from either an ARS/CEN or a 2μ plasmid under the control of a constitutively active Actin promoter.

Strains

To exclude any UPR signaling interference by the endogeneous Ire1p, a ire1Δ strain was used. The endogeneous Ire1 locus was substituted by homologous recombination with a kanamycine resistance cassette in JPY9, a α-strain auxotroph for HIS3, LTU2 LYS2, TRP1, URA3. In this strain, divergently oriented HIS3 and LacZ reporter genes containing an UPRE upstream of their promoters were integrated at the HIS3 locus. Upon transformation, cells were plated on minimal plates lacking the adequate amino acids;

β-Gal Assay and Growth Selection

To quantify the induction of the UPR signal cascade, LacZ reporter gene expression was measured by determining the activity of the LacZ genie-encoded β-Galactosidase (see Methods in yeast genetics, 2000 Edition, Cold Spring Harbor Laboratory Press, hereby incorporated by reference).

The activity of the HIS3 reporter gene was visualized by a growth selection assay. Upon transformation, cells were plated on plates lacking histidine. Only cells which activated the HIS3 reporter could grow. To set a growth threshold, cells were plated on -His plates containing 10, 30, 60, 90 and 120 millimolar 3AT. To induce the UPR, cells were grown in minimal media containing 1 μg/ml Tunicamycine.

Specific Interactions in the ER Lumen are Detectable

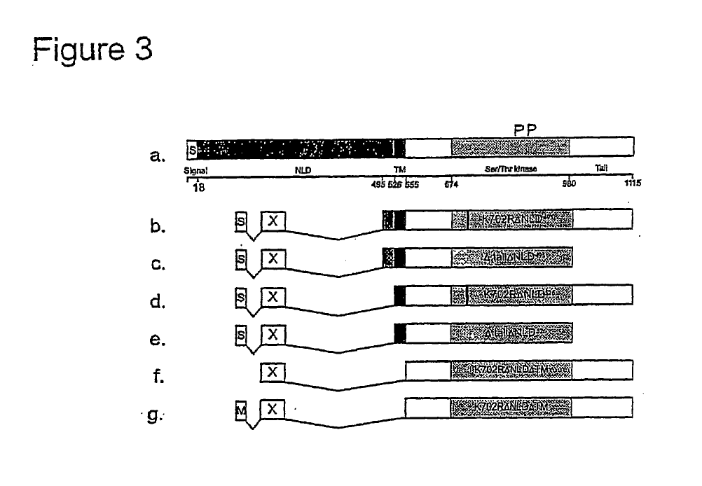

As described above, indication for activation of Ire1p upon dimerization comes from the observation that the two mutant Ire1p forms Ire1K702R and Ire1Δtail can functionally complement each other. In order to test whether dimerization is a prerequisite for this complementation, the Leucine-zipper of c-Jun (JunLZ) and the Leucine-zipper c-Fos (FosLZ) was inserted between a Suc2 signal sequence (S2ss) and different Ire1p C-terminal fragments, namely Ire1K702RΔNLD⁴⁹⁵, Ire1K702RΔNLD⁵²⁶, Ire1ΔtailΔNLD⁴⁹⁵, Ire1Δtail_NLD⁵²⁶ (K702R: point mutation K to R; Δtail: deletion of the C-terminal 133 Aa; ΔNLD⁴⁹⁵: truncation of the first 495 Aa of the NLD; ΔNLD⁵²⁶; complete truncation of the NLD. See FIG. 3. These fusion proteins were expressed from an ARS/CEN plasmid under the control of the constitutive actin promoter in an ire1Δ strain. UPR induction was quantified by measuring LacZ reporter gene expression controlled by a synthetic promoter containing the UPRE from the KAR2 promoter I/BiP expression is induced by UPR, REF1).

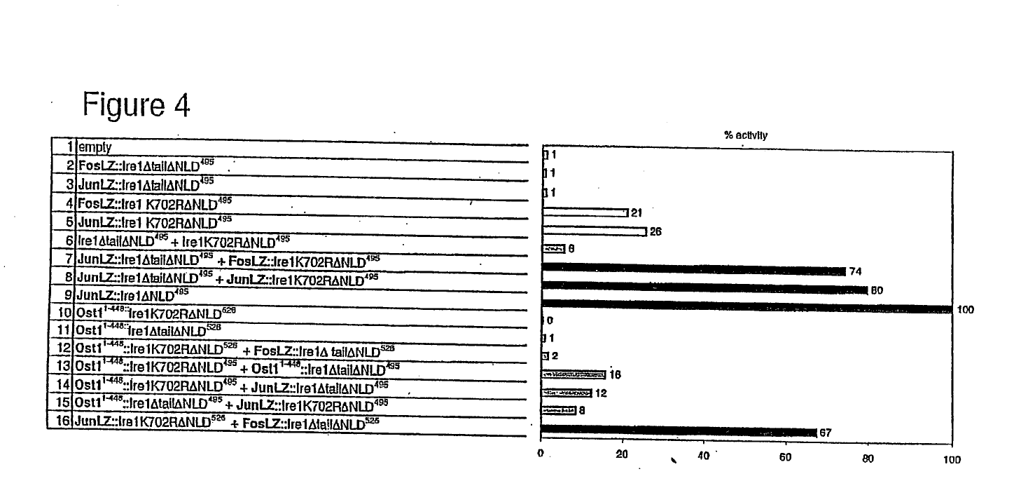

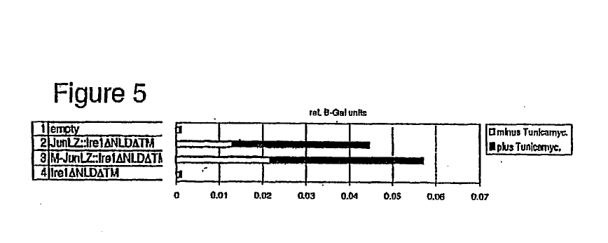

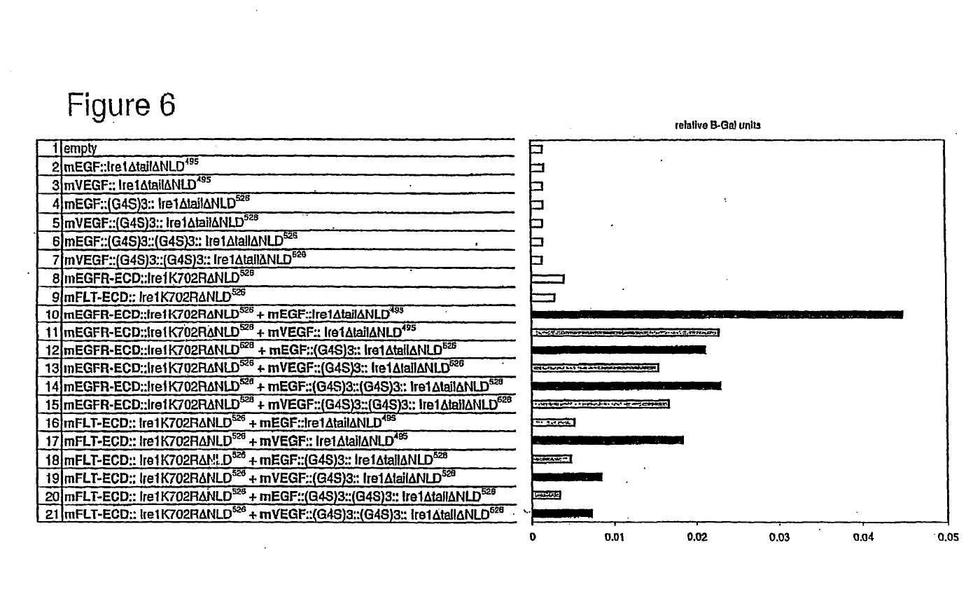

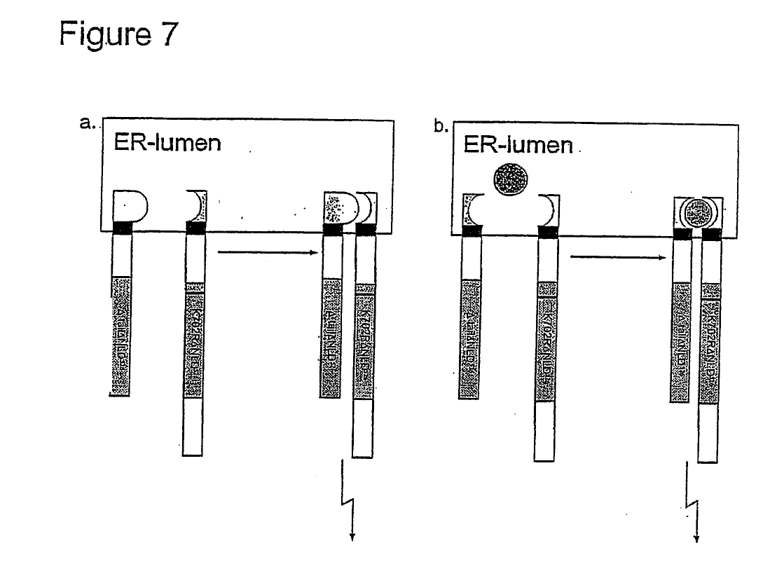

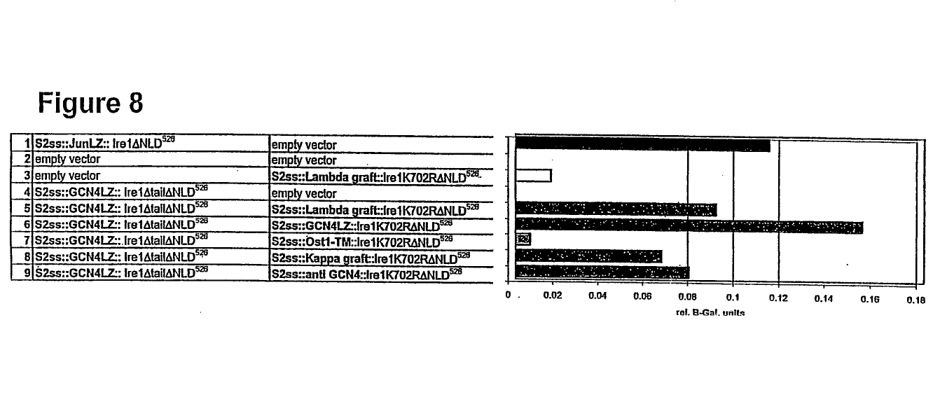

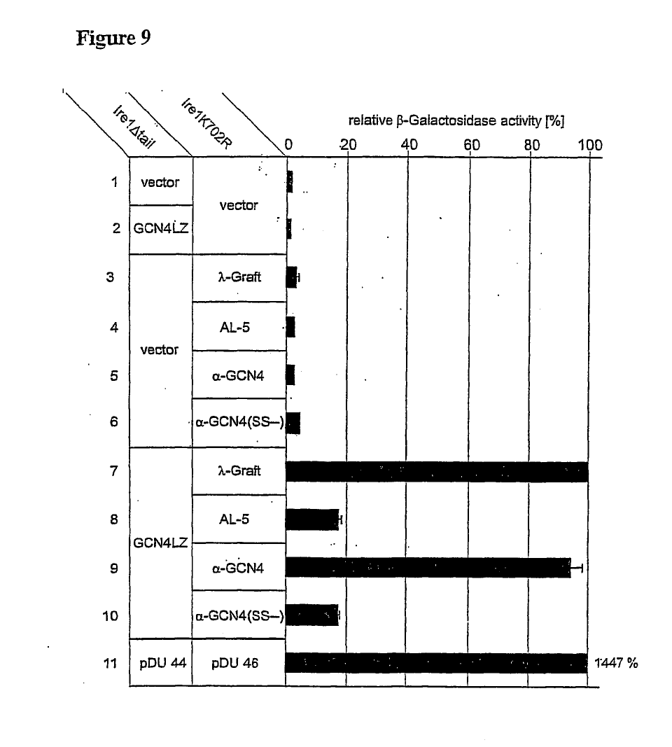

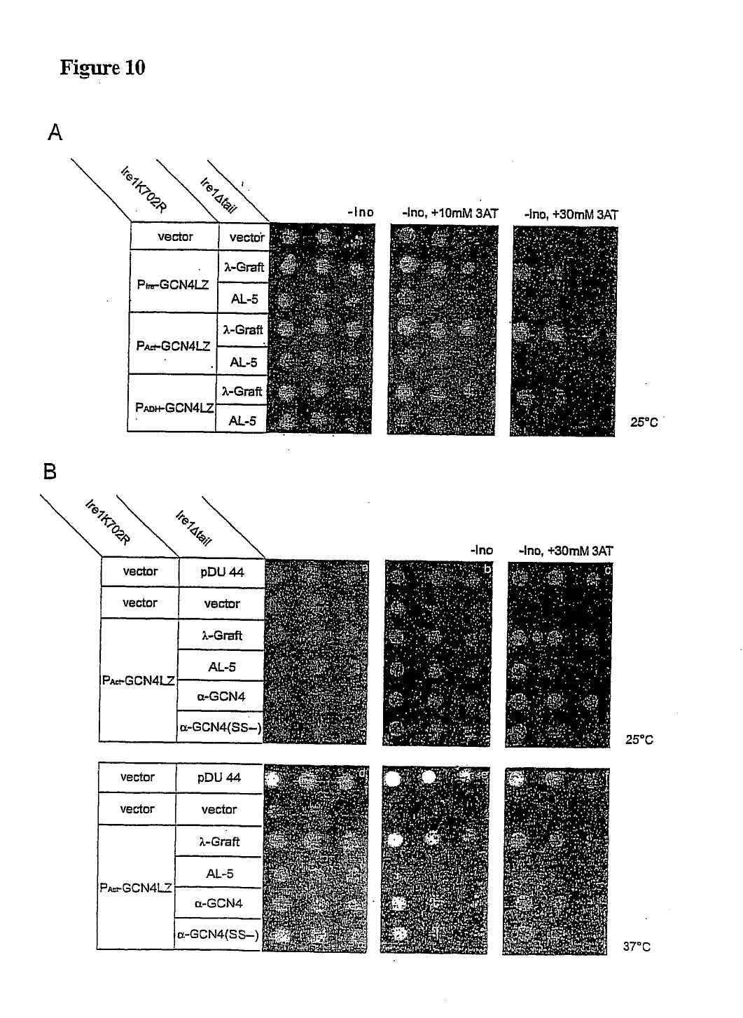

The constructs containing an Ire1 Δtail variant did not activate transcription above background, whereas those containing a Ire1K702R activated to about 30% of the level obtained with a wild-type Ire1p (FIG. 4 lines 2-4). In contrast, the same Ire1 mutants lacing a dimerization motif did not activate at all (FIG. 4 lines 5). Co-expression of the complementing mutants containing a dimerization motif activated reporter gene expression two to three folds the level reached by the expression of the K702R point mutation alone, and almost completely restored the activity of wild-type Ire1 C-terminus fused to JunLZ (FIG. 4 lines 7-9 and 16), which showed similar activity as full length Ire1p induced by Tunicamycin (data not shown)(Tunicamycin unduces the UPR by blocking the (N)-linked glycosylation, which leads to accumulation of unfolded proteins). Co-expression of the complementing mutants lacking a dimeization motif did not induce reporter gene expression.

In additional control experiment for the dependence of Ire1p activity on specific dimerization, the N-terminus of Ost1 (Ost1¹⁻⁴⁴⁸), an ER resident type I transmembrane protein, fused to either Ire1K702R or Ire1Δtail was co-expressed together with the construct mentioned above. Co-expression of Ost1¹⁴⁴⁸ fused to an Ire1 mutant together with JunLZ fused to the complementing mutant did not result in an increased activity (FIG. 4 lines 14-15), indicating that specific dimerization, and not just overexpression, leads to the synergistic effect of complementation.

### EXAMPLE 2

The Transmembrane Domain is not Necessary for the Ire1p activity

Ire1p is localized in the ER membrane and signals to the nucleus if unfolded proteins accumulate in the ER lumen. To test whether the association of Ire1p with the ER membrane is necessary for its fixation, JunLZ was fused with a Ire1p C-terminal fragment that lacks the transmembrane domain (TM) (Ire1ΔNLDΔTM). A myristoilation signal (M) was also added to the N-terminus of this fusion protein. FIG. 5 shows that, although the construct containing the MS had a higher activity then the one lacking the MS, both fusion proteins strongly activated Hac1p-dependent reporter gene expression.

Surprisingly, both Ire1 ΔNLDΔTM derivatives exhibiting dimerization ability were further activated by Tunicamycin. In contrast, the same cytoplasmic Ire1p fragment lacking a functional dimerization motif showed no constitutive activity and was also not inducible by Tunicamycin. These results indicate that, upon dimerization, the Ire1p C-terminal domain is able to signal from the cytosol and to sense the accumulation of unfolded proteins even when uncoupled from the ER-lumen. Thus, Ire1p might sense unfolded protein accumulation in the ER-lumen not only directly with its NLD but also by an additional signal in the cytosol. Since the Ire1 C-terminus is also able to activate reporter gene expression upon dimerization in the cytoplasm the system presented here can also be applied to detect protein-protein interactions in the cytoplasm.

### EXAMPLE 3

Ligands Bind Specifically to their Receptors in the ER Lumen

Although the growth hormone (GH) and the extracellular domain of its receptor can interact in a nuclear two-hybrid assay [11], the oxidizing environment of the secretory pathway and the extracellular matrix of living organisms can be a prerequisite for the proper folding and stability of many extracellular proteins, and might be obligatory for the function of other receptor-ligand pairs. By fusing the extracellular domains of receptors (mouse EGF receptor and mouse FLT1) to Ire1K702R⁵²⁶, and their specific ligands (mEGF and mVEGF) to Ire1tailΔNLD⁴⁹⁵, a system in which only co-expression of the appropriate receptor-ligand fusion protein pair should be able to activate the reporter genes was generated. In this system, potential autodimerization of ligand or receptor fusions cannot activate reporter gene expression because the receptors are fused to K702R mutants and the ligands to Δtail mutants. Binding of the ligand to its receptor induces dimerization of the two complementing Ire1 mutants (Ire1Δtail and Ire1K702R), thus activating the UPR signaling cascade.

Since the binding site of a ligand on its receptor varies from one ligand-receptor pair to the other, different linkers with variable length were inserted between the ligands and the Ire1p moiety. Thus, oligos coding for (G₄S)₃ were inserted between the ligand and the Ire1 part resulting in 4, 19, 34 amino acid spacers, or the first 31 amino acids of the lumenal part of Ire1p were used as a spacer. No significant difference between 4, 19 and 34 amino acid (G₄S)ₙ spacer was observed. When compared to these constructs the ones bearing the 31 amino acids of the Ire1p NLD showed the most prominent effect: the mouse EGF-Ire1tailΔNLD⁴⁹⁵ fusion co-expressed with, its receptor mEGFR-Ire1K702RΔNLD⁵²⁶ fusion activated the LacZ report genes two fold stronger than when co-expressed with mFLT1-Ire1K702RΔNLD⁵²⁶ (FIG. 6 lines 10 and 11). Mouse VEGF-Ire1ΔtailΔNLD⁴⁹⁵ fusion co-expressed with its receptor mFLT1-Ire1K702RΔNLD⁵²⁶ resulted in a three to four fold higher expression of LacZ that when co-expressed with mEGF-Ire1K702RΔNLD⁵²⁶ (FIG. 6 lines 16 and 17).

For the growth selection assay, cells expressing mFLT1 fusions and either mEGF or mVEGF ligands were inoculated in minimal medium. Dilution series of the liquid cultures were spotted onto minimal plates lacking histidine and containing 3AT, a competitive inhibitor of the HIS3 gene product. At 30 mM and higher 3AT concentrations, only cells expressing mFLT1-Ire1K702RΔNLD⁵²⁶ and mVEGF-Ire1Δt NLD⁴⁹⁵, but not cells expressing mEGF-Ire1ΔtailΔNLD⁴⁹⁵, were able to grow (data not shown).

### EXAMPLE 4

Read-Out:

The Saccharomyces cerevisiae unfolded protein stress sensor Ire1p is activated upon dimerization. Ire1p activation causes removal of the 252 nucleotide intron in the Hac1^{U}mRNA to produce the Hac1ⁱmRNA. This particular RNA splicing changes the open reading frame and allows the synthesis of a functional Hac1pⁱ. In the cellular system of the instant invention, Hac1pⁱ binds a UPRE in a synthetic promoter and activates transcription of the cognate selectable reporter genes (HIS3 and LacZ). A similar read out but with the mRNA of a synthetic transcription activator containing the Hac1 intron and other sequences necessary for the splicing reaction performed by Ire1p and tRNase is possible. In addition, by selecting a suitable reporter gene, growth selection of either agonists or antagonists can be performed.

The Two-Hybrid Approach:

Dimerization induced by any desired pair of interacting partners fused to the C-terminus of the two mutant forms Ire1K702R and Ire1Δtail is necessary and sufficient to induce the Ire1p activity and for further signaling leading to Hac1p-dependent gene activation (FIG. 7A).

The fact that Ire1NLDΔTM (lacking its transmembrane domain) retains signal capacity, allows its fusion to the C-terminus of full length proteins that harbor their own transmembrane domain(s) (e.g. receptors). This opens the possibility for screening full length cDNA libraries to identify transmembrane proteins binding a given extracellular protein.

The use of a cellular screening system to find targets for extracellular protein-protein interactions, for example single chain antibodies, is a reasonable alternative to the phage display method providing the advantage of circumventing the purification procedure of target proteins. A single chain library fused to the C-terminus of Ire1p coexpressed with the fusion of a target protein to the C-terminus of Ire1p enables the selection of binders.

Screening for Soluble Binders:

A major challenge for screening ligands that interact with receptors is the fact that these interactions occur in the extracellular environment. In cellular growth selection assays, all the cells in the neighborhood of a cell secreting a ligand which functionally interacts with a receptor would profit and thus grow, even if they express an unrelated ligand. Expression of the receptors and their soluble ligand in a closed compartment such as the ER, which provides the same properties as the extracellular space, should limit such background growth caused by the diffusion of the ligand. While the receptor chains are expressed as fusion with the Ire1p complementing mutants Ire1K702R and Ire1Δtail in the ER, a cDNA library can be expressed as such or fused to a ER retention signal. Ligands directed to the secretory pathway meet their receptors in the ER. Binding of a ligand to the ECD of its receptor leads to dimerization of the receptor chains which brings the Ire1p C-termini in close proximity and leads to UPR signaling and growth (i.e. expression of the detectable gene or reporter gene)(FIG. 7B).

### EXAMPLE 5

The interaction of three different single-chain antibodies directed against the leucine zipper of the yeast transcription factor GCN4 have been tested with their antigen (=GCN4 leucine zippper) in the Ire1 system. These single chain antibodies have been described (Wörn et al., J. Biol. Chem., 275, 2795-2803 (2000)) There it was shown that one of these three single chains (“anti-GCN4”, used in lane 9 in FIG. 8) was not stable under reducing conditions whereas the other two were stable. In the Wörn paper it was also shown that the “anti-GCN4”-single chain and the “lambda-graft” single chain (used in lane 3 and 5 in our figure) had higher in vitro affinity to the antigen than the “kappa-graft” single chain (used in lane 8 in our figure). These single chains were therefore fused to the Ire1 mutants and expressed in the ER (i.e., under oxidizing conditions). As is clear in FIG. 8, it was possible to (1) detect specific interactions between antigen and single chain antibody in vivo; (2) demonstrate that the “anti-GCN4” antibody is now stable; and (3) confirm the different affinities of the three single chain antibodies to their antigen in vivo (as they were determined in vitro in the Wörns paper).

This therefore demonstrates that the method of the instant invention allows for the screening of high affinity binding single chain antibodies against a given antigen under physiological, oxidizing conditions in vivo, for example, by screening a CDR-randomized single-chain library.

### EXAMPLE 6

### Detection of Single-Chain Fv-Antigen Interactions

To further evaluate the system we took advantage of the well characterized interaction between the three single chain Fv fragments, “anti-GNC4”, cysteine-free “anti-GCN4(SS--)” and “λ-Graft” and their epitope, the leucine zipper of GCN4 (GCN4LZ). As described by A. Wörn et.al, “anti-GCN4” has the highest affinity when measured in vitro with a K_{d} of (4.4+−0.1)×10⁻¹¹M followed by λ-Graft with a K_{d} of (3.8+−0.8)×10⁻¹⁰M. The ability of the cysteine-free “anti-GCN4(SS--)” to form disulfide bonds has been eliminated by mutating the four cysteine residues to either valine or alanine. The affinity of “anti-GCN4(SS--)” to the GCN4LZ is not measurable because the scFv is extremely prone to aggregation after purification and subsequent refolding. By measuring the onset of denaturation, both the “anti-GCN4” and the “λ-Graft” turned out to be stable, although the “λ-Graft” performed better. In agreement with these data, the intracellularly stable “λ-Graft” performed the best in a nuclear yeast two-hybrid assay whereas the wild-type “anti-GCN4” showed a very weak in vivo activity (about 5 times weaker reporter gene activity then λ-Graft). This is likely based on the failure to form disulfide bonds in the reducing intracellular environment. The mutated “anti-GCN4(SS--) did not bind to the antigen in this assay.

These scFvs were fused in our system between the SUC2 secretion signal (S2ss) and the C-terminal moiety of Ire1K702ΔNLD⁴⁹⁵. The leucine-zipper of GCN4 was fused between S2ss and Ire1ΔtailΔNLD⁴⁹⁵ (FIG. 2) in order to prevent activation of the UPR signaling cascade due to the strong homodimerization activity of GCN4LZ. To minimize unspecific dimerization due to overexpression of the chimeras, we expressed the scFvs from the very weak IRE1 promoter whose activity on the plasmids used in this experiment is about 7 times weaker than that of the truncated ADH promoter and as much as 140 times weaker than the actin promoter. The potential of the scFvs to bind GCN4LZ, thus dimerizing the complementing Ire1 C-terminal moieties and, as a consequence, activating the UPR signalling cascade, was monitored by measuring the β-galactosidase reporter gene activity under the control of 1×UPRE. The epitope fused to Ire1Δtail⁴⁹⁵⁻⁹⁸² in contrast was expressed from the actin promoter. None of the constructs showed any activity if expressed alone (FIG. 9, lines 2-6). The λ-Graft strongly activated UPR signaling and reporter gene activity if co-expressed with its epitope. In contrast, the non-selective AL-5 showed only a very low level of induction (FIG. 9, lines 7 and 8). Likewise the anti-GCN4 wt scFv which was shown to have a high binding affinity induced reporter gene activity strongly, while the cysteine-free anti GCN4(SS--) activated the system to the same low extent as the unspecific AL-5 (see FIG. 9). Because the formation of disulfide bonds in the oxidizing environment is a prerequisite for proper folding of the characteristic immunoglobulin domains, the mutations of “anti-GCN4(SS--) may impair the conformational stability of this scFv and abolish binding to the epitope. In contrast, the wild-type “anti-GCN4” performs as well as the intracellularly more stable “λ-Graft”. The fact that all the signals of all the single-chains mentioned above appear in a western blot in a comparable intensity is in agreement with the assumption that differences in conformational stability rather than protein stability cause the differences in reporter gene activation between the wild-type and the mutated anti-GCN4 scFv. This data demonstrates that a specific interaction between two proteins fused to the complementing mutants of Ire1p is required for UPR signalling.

### EXAMPLE 7

### Specific Interactions are Selectable on Plates Lacking Histidine

To select cells expressing two interacting proteins fused to the complementing mutants of Ire1p, DIKU1 cells were transformed with Ars/Cen plasmids expressing the single-chains λ-Graft and AL-5 from an IRE1 promoter. The DIKU1 strain expresses the HIS3 and LacZ reporter genes from a bidirectional promoter under the control of 1×UPRE. The GCN4LZ epitope was expressed either from an actin, a truncated ADH or ah IRE1 promoter. Exponentially-growing cell cultures were spotted on selective plates lacking histidine, tryptophane and leucine and containing 0, 10, 30, 60 and 90 mM 3AT (3-Aminotriazol) which is a competitive inhibitor of the HIS3 gene product. Independently of the promoter expressing the epitope, cells transformed with empty vectors or the non-specific AL-5 stopped growing at 3AT concentrations of 30 mM (FIG. 10A), whereas cells expressing the λ-Graft still grew at concentrations as high a 90 mM 3AT. The most pronounced effect was observed with cells expressing the GCN4LZ from an actin promoter at 30 mM 3AT (FIG. 10A).

### EXAMPLE 8

### Growth Selection on Inositol-Lacking Plates at Elevated Temperature

To monitor the contribution to growth selection of the HIS3 transcriptional read-out and the UPR induced inositol synthesis and the temperature tolerance upon Ire1p dimerization, the Δire1Δder1 strain DIKU1 was transformed with Ars/Cen plasmids expressing the GCN4LZ from an actin promoter and the single-chains from an IRE1 promoter. Overnight cultures were spotted on agar plates. The control plates lacked histidine, leucine and tryptophane whereas the selective plates additionally labeled inositol and contained 0 or 30 mM 3AT. All the plates were incubated at either 25° C. or at 37° C. As expected, on the control plates at 25° C. all the transformants grew well (FIG. 10B a). While at single read-out conditions (plates lacking inositol or incubation of non-selective plates at 37° C.) only the vector controls showed growth retardation (FIG. 10B b, d), the combination of elevated temperature and inositol deprivation allowed a clear selection between the GCN4LZ binder λ-Graft and the non binder AL-5 (FIG. 10B e). Addition of 30 mM 3AT further enhanced the stringency of the growth selection at all conditions (FIG. 10B c, f). In contrast, it was possible to discriminate between the anti-GCN4 and the mutated cys-free anti-GCN4(SS--) at 25° C. on 3AT plates lacking inositol, but not on 37° C. at any condition (FIG. 10B d, e, f). Although the mutation in cys-free anti-GCN4 causes a change in the conformational structure of the Ig domain, it appears that the protein is still stable at 30° C. (compare β-galactosidase values in FIG. 9). At the non-permissive 37° C. the protein likely unfolds and aggregates leading to dimerization of the Ire1K702R moiety and allowing growth at elevated temperature. However, in the DIKU1 strain these aggregates cannot be degraded through the ERAD due to mutation of the DER1 gene. In the ERAD wild-type but otherwise identical strain IKU1, anti-GCN4 was selectable by growth from the cys-free anti-GCN4(SS--) at 37° C. on 3AT plates lacking inositol.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims.

### REFERENCES

1. Ellgaard, L., M. Molinari, and A. Helenius, Setting the standards: quality control in the secretory pathway. Science, 1999. 286(5446): p. 1882-8.
2. Abkevich, V. I. and E. I. Shakhnovich, What can disulfide bonds tell us about protein energetics function and folding: simulations and bioninformatics analysis. J Mol Biol, 2000. 300(4): p. 975-85.
3. Rudd, P. M., et al., Roles for glycosylation of cell surface receptors involved in cellular immune recognition. J Mol Biol, 1999. 293(2): p. 351-66.
4. Friedlander, R., et al., A regulatory link between ER-associated protein degradation and the unfolded-protein response. Nat Cell Biol, 2000. 2(7): p. 379-84.
5. Travers, K. J., et al., Functional and genomic analyses reveal an essential coordination between the unfolded protein response and ER-associated degradation. Cell. 2000. 101(3): p. 249-58.
6. Cox, J. S., C. E. Shamu, and P. Walter, Transcriptional induction of genes encoding endoplasmic reticulum resident proteins requires a transmembrane protein kinase. Cell, 1993. 73(6): p. 1197-206.
7. Liu C. Y., M. Schroder, and R. J. Kaufman, Ligand-independent dimerization activates the stress response kinases IRE1 and PERK in the lumen of the endoplasmic reticulum. J Biol Chem, 2000. 275(32): p. 24881-5.
8. Sidrauski, C. and P. Walter, The transmembrane kinase Ire1p is a site-specific endonuclease that initiates mRNA splicing in the unfolded protein response. Cell, 1997. 90(6): p. 1031-9.
9. Gonzalez, T. N., et al., Mechanism of non-spliceosomal mRNA splicing in the unfolded protein response pathway. Embo J, 1999. 18(11): p. 3119-32.
10. Kohno, K., et al., The promoter region of the yeast KAR2 (BiP) gene contains a regulatory domain that responds to the presence of unfolded proteins in the endoplasmic reticulum. Mol Cell Biol, 1993. 13(2): p. 877-90.
11. Ozenberger, B. A. and K. H. Young, Functional interaction of ligands and receptors of the hematopoietic superfamilly in yeast. Mol Endocrinol, 1995. 9(10): p. 1321-9.

1. A method for transferring a phosphate group to a first hybrid protein, the method comprising:
(a) providing a first chimeric gene that is capable of being expressed in the host cell, the first chimeric gene comprising a DNA sequence that encodes a first hybrid protein, the first hybrid protein comprising:
(i) a first Ire1 like polypeptide with an inactive or absent native kinase domain; and
(ii) a first test protein or fragment thereof that is to be tested for interaction with at least one second test protein or fragment thereof;
(b) providing a second chimeric gene that is capable of being expressed in the host cell, the second chimeric gene comprising a DNA sequence that encodes a second hybrid protein, the second hybrid protein comprising:
(i) a second Ire1 like polypeptide which lacks the Ire1 dimerization ability but possesses a kinase domain; and
(ii) a second test protein or fragment thereof that is to be tested for interaction with the first test protein or fragment thereof;
wherein interaction between the first test protein and the second test protein in the host cell results in the dimerization of the first hybrid protein and second hybrid protein, which results in transfer of a phosphate group to the first hybrid protein;
(c) introducing the first chimeric gene and the second chimeric gene into the host cell;
(d) subjecting the host cell to conditions under which the first hybrid protein and the second hybrid protein are expressed in sufficient quantity for the dimerization of the first hybrid protein and second hybrid protein; and
(e) subjecting the host cell to conditions under which the second hybrid protein catalyzes the transfer of a phosphate group to the first hybrid protein
wherein the host cell is:
(a) a Hac⁻ cell that comprises a synthetic signaling transcription factor;
(b) a cell that is both Ire1⁻ and ERAD⁻ wherein the cell is grown at elevated temperatures; or
(c) grown on media lacking inositol.

2. A method for transferring a phosphate group to a first hybrid protein, the method comprising:
(a) providing a first chimeric gene that is capable of being expressed in the host, cell, the first chimeric gene comprising a DNA sequence that encodes a first hybrid protein, the first hybrid protein comprising:
(i) an Ire1 like polypeptide with an inactive or absent native kinase domain; and
(ii) a first test protein or fragment thereof that is to be tested for interaction with at least one third test protein or fragment thereof;
(b) providing a second chimeric gene that is capable of being expressed in the host cell, the second chimeric gene comprising a DNA sequence that encodes a second hybrid protein, the second hybrid protein comprising:
(i) an Ire1 like polypeptide which lacks the Ire1 dimerization ability but possesses a kinase domain; and
(ii) a second test protein or fragment thereof that is to be tested for interaction with the at least one third test protein or fragment thereof;
wherein a simultaneous interaction between the third test protein and both the first test protein and the second test protein in the host cell results in the dimerization of the first hybrid protein and second hybrid protein, which results in transfer of a phosphate group to the first hybrid protein;
(c) introducing the first chimeric gene and the second chimeric gene into the host cell;
(d) subjecting the host cell to conditions under which the first hybrid protein and the second hybrid protein and the third test protein are expressed in sufficient quantity for the dimerization of the first hybrid protein and second hybrid protein; and
(e) subjecting the host cell to conditions under which the second hybrid protein catalyzes the transfer of a phosphate group to the first hybrid protein.

3. A method for detecting an interaction between a first test protein and a second test protein, the method comprising:
(a) providing a host cell;
(b) providing a first chimeric gene that is capable of being expressed in the host cell, the first chimeric gene comprising a DNA sequence that encodes a first hybrid protein, the first hybrid protein comprising:
(i) an Ire1 like polypeptide with an inactive or absent native kinase domain; and
(ii) a first test protein or fragment thereof that is to be tested for interaction with at least one second test protein or fragment thereof;
(c) providing a second chimeric gene that is capable of being expressed in the host cell, the second chimeric gene comprising a DNA sequence that encodes a second hybrid protein, the second hybrid protein comprising:
(i) an Ire1 like polypeptide which lacks the Ire1 dimerization ability but possesses a kinase domain; and
(ii) a second test protein or fragment thereof that is to be tested for interaction with the first test protein or fragment thereof;
(d) introducing the first chimeric gene and the second chimeric gene into the host cell; and
(e) subjecting the host cell to conditions under which the first hybrid protein and the second hybrid protein are expressed in sufficient quantity that the first hybrid protein and second hybrid protein dimerize and the second hybrid protein catalyzes the transfer of a phosphate group to the first hybrid protein wherein phosphorylation of the first hybrid protein results in a signal which can be detected wherein the host cell is:
(d) a Hac⁻ cell that comprises a synthetic signaling transcription factor;
(e) a cell that is both Ire1⁻ and ERAD⁻ wherein the cell is grown at elevated temperatures; or
(f) grown on media lacking inositol.

4. A method for detecting an interaction between a first test protein and a second test protein, the method comprising:
(a) providing a host cell containing a detectable gene(s), wherein the detectable gene(s) expresses a detectable protein(s) when the detectable gene(s) is activated by a signaling transcription factor, when the signaling transcription factor is in sufficient proximity to the detectable gene;
(b) providing a first chimeric gene that is capable of being expressed in the host cell, the first chimeric gene comprising a DNA sequence that encodes a first hybrid protein, the first hybrid protein comprising:
(i) an Ire1 like polypeptide with an inactive or absent native kinase domain; and
(ii) a first test protein or fragment thereof that is to be tested for interaction with at least one second test protein or fragment thereof;
(c) providing a second chimeric gene that is capable of being expressed in the host cell, the second chimeric gene comprising a DNA sequence that encodes a second hybrid protein, the second hybrid protein comprising:
(i) an Ire1 like polypeptide which lacks the Ire1 dimerization ability but possesses a kinase domain; and
(ii) a second test protein or fragment thereof that is to be tested for interaction with the first test protein or fragment thereof;
wherein interaction between the first test protein and the second test protein in the host cell results in the dimerization of the first hybrid protein and second hybrid protein which further results in the transfer of a phosphate group to the first hybrid protein catalyzed by the kinase domain of the second hybrid protein;
(d) introducing the first chimeric gene and the second chimeric gene into the host cell;
(e) subjecting the host cell to conditions under which the first hybrid protein and the second hybrid protein are expressed in sufficient quantity that the first hybrid protein and second hybrid protein dimerize; and
(f) subjecting the host cell to conditions under which the second hybrid protein catalyzes the transfer of a phosphate group to the first hybrid protein;
(g) subjecting the host cell to conditions under which phosphorylation of the first hybrid protein results in activation of the signaling transcription factor;
(h) subjecting the host cell to conditions under which the activated signaling transcription factor is able to be in sufficient proximity to the detectable gene(s) to result in expression of the detectable protein(s); and
(i) determining whether the detectable gene(s) has been expressed to a degree greater than expression in the absence of an interaction between the first test protein and the second test protein
wherein the host cell is a Hac⁻ cell that comprises a synthetic signaling transcription factor.

5. A method for identifying the DNA of interacting proteins, comprising performing steps (a)-(i) according to claim 4 and further comprising:
(j) identifying the chimeric genes present in host cells which express the detectable gene to a degree greater than expression in the absence of an interaction between the first test protein and the second test protein.

6. The method according to claim 1, wherein the host cell is selected from the group consisting of:
(a) Saccharomyces cerevisiae;
(b) Mammalian cells;
(c) Eukaryotic cells; and
(d) Prokaryotic cells.

7. The method according to claim 1, wherein the first hybrid protein or the second hybrid protein is encoded on a library of plasmids containing DNA inserts, derived from the group consisting of genomic DNA, cDNA and synthetically generated DNA.

8. The method according to claim 1, wherein the first test protein or second test protein or both the first and second test proteins are derived from the group consisting of
(a) bacterial proteins;
(b) viral proteins;
(c) oncogene-encoded proteins;
(d) eukaryotic proteins
(e) plant proteins;
(f) yeast proteins;
(g) orphan receptors;
(h) antibodies;
(i) antigens;
(j) ligands;
(k) any transmembrane protein;
(l) any cell surface protein;
(m) any extracellular protein;
(n) any protein expressed in the secretory pathway; and
(O) any intracellular protein.

9. The method according to claim 1, wherein the chimeric genes are introduced into the host cell in the form of plasmids.

10. The method according to claim 1, wherein the first chimeric gene is integrated into the chromosomes of the host cell.

11. The method according to claim 1, wherein the first chimeric gene is integrated into the chromosomes of the host cell and the second chimeric gene is introduced into the host cell as part of a plasmid.

12. The method according to claim 1, wherein the Ire1 like polypeptide is selected from the group consisting of:
(a) Ire1 homologs;
(b) Ire1 derived polypeptides; and
(c) Ire1 polypeptides.

13. The method according to claim 1, wherein the Ire1 like polypeptide with the inactive or absent native kinase domain is any complementable kinase mutant of Ire1.

14. The method according to claim 13, wherein the Ire1 derived polypeptide with the inactive or absent native kinase domain is selected from the group consisting of:
(a) Ire1K702R;
(b) Ire1 K702RΔNLD⁴⁹⁵;
(c) Ire1 K702RΔNLD⁵²⁶
(d) Ire1 K702RΔNLDΔTM;
(e) a protein comprising the wild type cytoplasmic portion of Ire1;
(f) Myristoylated Ire1 K702RΔNLDΔTM; and
(g) Any fragment or derivative of (a)-(e) capable of complementing an Ire1 mutant which lacks dimerization ability.

15. The method according to claim 1, wherein the Ire1 derived polypeptide which lacks the Ire1 dimerization ability but possesses a kinase domain is any complementable dimerization mutant of Ire1.

16. The method according to claim 15, wherein the Ire1 derived polypeptide which lacks the Ire1 dimerization ability but possesses a kinase domain is selected from the group consisting of:
(a) Ire1Δtail;
(b) Ire1ΔtailΔNLD⁴⁹⁵;
(c) Ire1 ΔtailΔNLD⁵²⁶;
(d) Ire1ΔtailΔTM;
(e) myristoylated Ire1ΔtailΔTM; and
(f) Any fragment or derivative of (a)-(e) capable of complementing an Ire1 mutant which lacks dimerization ability.

17. The method according to claim 1, wherein the interaction between the first test protein and second test protein occurs in the cytoplasm, on the cell surface or anywhere in the secretory pathway.

18. The method according to claim 1, wherein either the first test protein or the second test protein or both the first test protein and the second test protein are expressed such that they remain in the endoplasmic reticulum.

19. The method according to claim 1, wherein either the first test protein or the second test protein or both the first test protein and the second test protein are full length proteins.

20. The method according to claim 1, wherein either the first test protein or the second test protein or both the first test protein and the second test protein possess transmembrane domains.

21. The method according to claim 1, wherein either the first test protein or the second test protein is a single chain antibody.

22. The method according to claim 4, wherein the detectable gene is the LacZ gene.

23. The method according to claim 4, wherein the detectable gene is the HIS3 gene.

24. The method according to claim 4, wherein the detectable genes are the LacZ gene and the HIS3 gene.

25. The method according to claim 4, wherein the detectable gene is selected from the group consisting of:
(a) CAT (chloramphenicol acetyltransferase);
(b) GAL (β-galactosidase);
(c) GUS (β-glucuronidase);
(d) URA3;
(e) LUC (luciferase); and
(f) GFP (green fluorescent protein).

26. The method according to claim 4, wherein the detectable gene is in proximity to an Unfolded Protein Response Element (UPRE).

27. The method according to claim 26, wherein the UPRE is the yeast UPRE.

28. The method according to claim 26, wherein the UPRE is an ERST.

29. A chimeric gene comprising a DNA sequence that encodes a hybrid protein, the hybrid protein comprising:
(a) an Ire1 like polypeptide with an inactive or absent native kinase domain; and
(b) a test protein or fragment thereof.

30. A chimeric gene comprising a DNA sequence that encodes a hybrid protein, the hybrid protein comprising:
(a) an Ire1 like polypeptide which lacks the Ire1 dimerization ability but possesses a kinase domain; and
(b) a test protein or fragment thereof.

31. The chimeric gene according to claim 29, wherein the Ire1 like polypeptide is selected from the group consisting of:
(a) Ire1 homolog polypeptides;
(b) Ire1 derived polypeptides; and
(c) Ire1 polypeptides.

32. The chimeric gene of claim 29, wherein the Ire1 like polypeptide is any complementable kinase mutant of Ire1.

33. The chimeric gene of claim 29, wherein the Ire1 like polypeptide is selected from the group consisting of:
(a) Ire1K702R;
(b) Ire1 K702RΔNLD⁴⁹⁵;
(c) Ire1 K702RΔNLD⁵²⁶
(d) Ire1 K702RΔNLDΔTM;
(e) Myristoylated Ire1 K702RΔNLDΔTM; and
(f) Any fragment or derivative of (a)-(e) capable of complementing an Ire1 mutant which lacks dimerization ability.

34. The chimeric gene of claim 30, wherein the Ire1 like polypeptide is any complementable dimerization mutant of Ire1.

35. The chimeric gene of claim 30, wherein the Ire1 like polypeptide is selected from the group consisting of:
(a) Ire1Δtail;
(b) Ire1ΔtailΔNLD⁴⁹⁵;
(c) Ire1ΔtailΔNLD⁵²⁶;
(d) Ire1ΔtailΔTM;
(e) myristoylated Ire1ΔtailΔTM; and
(f) Any fragment or derivative of (a)-(e) capable of complementing an Ire1 mutant which lacks dimerization ability.

36. A protein encoded by the chimeric gene of claim 29.

37. A protein encoded by the chimeric gene of claim 30.

38. A vector comprising the chimeric gene of claim 29.

39. A vector comprising the chimeric gene of claim 30.

40. A vector comprising a DNA sequence capable of encoding an Ire1 like polypeptide wherein the native kinase domain of the polypeptide is inactive or:
absent and further comprising a cloning site which allows for the construction of the chimeric gene of claim 29.

41. A vector comprising a DNA sequence capable of encoding an Ire1 like polypeptide wherein the polypeptide lacks the Ire1 dimerization ability but possesses a kinase domain and further comprising a cloning site which allows for the construction of the chimeric gene of claim 30.

42. A host cell comprising:
(a) the chimeric gene of claim 29;
(b) the chimeric gene of claim 30; or
(c) both the chimeric gene of claiim 29 and the chimeric gene of claim 30.

43. A kit comprising any one or more of the following:
(a) the chimeric gene of claim 29;
(b) the chimeric gene of claim 30;
(c) the vector of claim 38;
(d) the vector of claim 39;
(e) the vector of claim 40;
(f) the vector of claim 41; and
(g) the host cell of claim 42.

44. A method for identifying an inhibitor of an interaction between two proteins comprising:
(a) providing a host cell;
(b) providing a first chimeric gene that is capable of being expressed in the host cell, the first chimeric gene comprising a DNA sequence that encodes a first hybrid protein, the first hybrid protein comprising:
(i) an Ire1 like polypeptide with an inactive or absent native kinase domain; and
(ii) a first test protein or fragment thereof that is to be tested for interaction with at least one second test protein or fragment thereof;
(c) providing a second chimeric gene that is capable of being expressed in the host cell, the second chimeric gene comprising a DNA sequence that encodes a second hybrid protein, the second hybrid protein comprising:
(i) an Ire1 like polypeptide which lacks the Ire1 dimerization ability but possesses a kinase domain; and
(ii) a second test protein or fragment thereof that is to be tested for interaction with the first test protein or fragment thereof;
(d) introducing the first chimeric gene and the second chimeric gene and an inhibitor candidate into the host cell;
(e) subjecting the host cell to conditions under which the first hybrid protein and the second hybrid protein are expressed in sufficient quantity that the first hybrid protein and second hybrid protein could, in the absence of an inhibitor, dimerize wherein dimerization would cause the second hybrid protein to catalyze the transfer of a phosphate group to the first hybrid protein wherein phosphorylation of the first hybrid protein results in a signal which can be detected;
(i) determining whether the signal is stronger or weaker than the signal in the absence of the agent; and
(j) identifying the agent used as the inhibitor when the detectable gene has been expressed to a degree less than expression in the absence of the agent.

45. The method of claim 44, wherein the agent is selected from the group consisting of:
(a) proteins;
(b) small molecules;
(c) chemical compounds;
(d) peptides; and
(e) natural molecules.

46. The method according to claim 44, wherein the signal comprises a signaling transcription factor interacting with a detectable gene.

47. The method according to claim 4, wherein the signaling trancription factor is a Hac1 like polypeptide.

48. The method according to claim 4 wherein the transcription factor is a synthetic transcriptional activator.

49. The method according to claim 46, wherein the Ire1 like polypeptides are selected from the group consisting of:
(a) Ire1 homolog polypeptides;
(b) Ire1 derived polypeptides; and
(c) Ire1 polypeptides.

50. The method according to claim 48, wherein the synthetic transcriptional activator is translated from RNA that is spliced by Ire1 like RNase activity.

51. The method according to claim 48, wherein the host cell does not express endogenous Hac1 like polypeptides.

52. The host cell of claim 42, wherein the host cell does not produce endogenous Ire1 like polypeptides.

53. The method of claim 6, wherein the host cell does not produce endogenous Ire1 like polypeptides.

54. The method of claim 2, wherein the third test protein is a single chain antibody.
